(19) United States
(12) Patent Application Publication
(10) Pub. No.: US 2025/0241984 A1
(43) Pub. Date: Jul. 31, 2025

(54) TARGETTING ACTIVITY OF THE MITF FAMILY AS A THERAPEUTIC FOR REGULATING GENE EXPRESSION NETWORKS IMPLICATED IN B CELL HOMEOSTASIS, GERMINAL CENTER RESPONSES, AND TOLERANCE

(71) Applicant: The Research Foundation for the State University of New York, Albany, NY (US)

(72) Inventors: Christopher Roman, Brooklyn, NY (US); Abhimanyu Amarnani, New York City, NY (US)

(21) Appl. No.: 19/038,490

(22) Filed: Jan. 27, 2025

Related U.S. Application Data
(60) Provisional application No. 63/625,680, filed on Jan. 26, 2024.

(51) Int. Cl. A61K 38/17; A61K 48/00; C12N 15/86; C12Q 1/6883

(52) U.S. Cl. CPC A61K 38/1709; A61K 48/005; C12N 15/86; C12Q 1/6883; C12N 2740/15043; C12Q 2600/158

(57) ABSTRACT

The microphthalmia transcription factor Mitf has been shown to regulate B cell activation and tolerance. However, the underlying B cell-specific mechanisms responsible, and those that distinguish Mitf from closely related Mitf/TFE (MiT) transcription factors Tfe3, Tfeb, and Tfec, remain obscure. Two complementary mouse models of Mitf and B-cell specific MiT family deficiency were used to define how MiT family candidate target genes and pathway dysregulation can occur in pathogenic B cells due to Tfec-like overexpression and subsequent loss-of-protective functions of Mitf for autoimmune tolerance. These findings underscore the critical role of Mitf in maintaining B cell homeostasis and self-tolerance, and highlight the potential for therapeutics to either decrease the functionality of Tfec and/or increase the functionality of Mitf to treat autoimmune diseases.

### CROSS-REFERENCE TO RELATED APPLICATIONS

This Application claims the benefit of the filing date of U.S. Provisional Application No. 63/625,680 filed Jan. 26, 2024. The content of this earlier filed application is hereby incorporated by reference herein in its entirety.

### BACKGROUND OF THE INVENTION

### Field of the Invention

Autoimmune diseases affect millions of people worldwide, with over 80 known conditions causing significant morbidity and mortality. Current treatments for autoimmune disorders often involve broad immunosuppression, which can lead to serious side effects and increased susceptibility to infections and malignancies. While biological drugs and small molecule inhibitors targeting inflammatory cytokines, immune cells, and intracellular kinases have improved treatment options, many patients still do not respond adequately to existing therapies. There is an urgent need for novel therapeutic approaches that can achieve sustained, treatment-free remission while minimizing long-term toxicities. This invention relates to novel methods for treating autoimmune diseases and eliminate self-reactive immune cells while preserving normal immune function. The proposed therapy builds upon recent advances in cell technology, which have shown promise. The disclosed methods aim to provide a more precise and effective treatment option for various autoimmune conditions, potentially transforming patient outcomes and quality of life.

### BRIEF SUMMARY OF THE INVENTION

Embodiments disclosed herein are directed to a method for reducing the activity of the Tfec transcription factor in B lymphocytes. According to one embodiment the method comprises the step of increasing the activity of other MiT family transcription factors Mitf, Tfeb, and Tfe3. According to embodiments, the method is for the treatment of B cell-mediated autoimmune disorders.

Other embodiments disclosed herein are directed to a method for restoring immune tolerance in self-reactive B cells by regulating the balance between Mitf and Tfec transcription factors, comprising either decreasing Tfec activity by directly targeting Tfec DNA/RNA using, for example, engineered zinc finger proteins, CRISPR-guided base editing, transcription activator-like effector nucleases (TALENs), or RNA-targeted therapeutics using ADARs, or indirectly targeting Tfec protein using structures that block its function or promote its degradation, or increasing Mitf expression and translation by using engineered zinc finger proteins to activate the Mitf gene promoter, using CRISPR-guided base editing to introduce beneficial mutations in the Mitf gene regulatory regions, or decreasing proteosomal degradation of Mitf, thereby restoring the ability of Mitf (with Tfe3 and/or Tfeb) to maintain immune tolerance in autoreactive B cells.

According to other embodiments disclosed herein are methods for treating autoimmune diseases in a subject in need thereof, said auto immune disease include systemic lupus erythematosus, rheumatoid arthritis, Sjogren's syndrome, and multiple sclerosis, the method comprising administering to the subject a therapeutically effective amount of an agent that inhibits miR-148a in B lymphocytes of the subject, thereby increasing Mitf expression and restoring the ability of Mitf, in conjunction with Tfe3 and/or Tfeb, to maintain immune tolerance.

According to other embodiments disclosed herein are methods for regulation of homeostasis and immunological tolerance in B lymphocytes, wherein the method comprises the step of augmenting the functional capacity of MiT family transcription factors, Mitf, Tfeb, and/or Tfe3.

According to other embodiments are the methods for regulation of homeostasis and immunological tolerance in B lymphocytes. The method comprises the step of augmenting the functionality of one or more MiT family transcription factors selected from the group consisting of Mitf, Tfeb, and Tfe3 and the functional capacity comprises one or more of: DNA binding activity; transcriptional activation of target genes; protein stability; and formation of functional transcriptional complexes. According to embodiments the regulation of homeostasis and immunological tolerance in B lymphocytes provides therapeutic management of B cell mediated autoimmune disorders systemic lupus erythematosus, rheumatoid arthritis, Sjogren's syndrome, and multiple sclerosis.

According to another embodiment, therapeutic management comprises reducing autoantibody production, immune complex formation, inflammatory cytokine production, and/or ectopic lymphoid structure formation, as well reducing B cell infiltration into exocrine glands, reducing autoantibody production, and/or reducing glandular inflammation; and multiple sclerosis, and/or reducing central nervous system inflammation.

Embodiments disclosed herein are also directed to methods of diagnosing patients exhibiting altered B lymphocyte functionality. The methods comprise: quantification of transcription factor levels of Tfec or decreased levels of Mitf, Tfeb, and/or Tfe3 in B cells and/or B cell subsets (e.g. double negative 2, memory, regulatory, aging-associated, and plasmablast B cells). The methods further comprise: (1) obtaining a biological sample from the patient, (2) isolating B lymphocytes from the sample, (3) measuring the expression levels of Tfec, Mitf, Tfeb, and Tfe3 (MiT family) in the isolated B cells and/or B cell subsets using techniques such as quantitative PCR, Western blotting, or immunofluorescence, among other techniques, and (4) comparing these levels to established normal ranges for the MiT family in healthy control and patients with B cell mediated autoimmune disorders, systemic lupus erythematosus, rheumatoid arthritis, Sjogren's syndrome, and multiple sclerosis.

Further embodiments are directed to a method for modulating B cell homeostasis and tolerance, comprising: i) identifying a subject with impaired Mitf function, ii) introducing a nucleic acid molecule encoding a functional Mitf protein into B cells of the subject, and iii) expressing the Mitf protein in the B cells to regulate gene expression networks implicated in B cell homeostasis and tolerance. Other embodiments disclosed herein are directed to a composition for treating autoimmune diseases associated with impaired Mitf function, comprising: i) a nucleic acid molecule encoding a functional Mitf protein, ii) a vector for delivering the nucleic acid molecule to B cells, iii) optionally, a pharmaceutical carrier. The composition further comprises an immunosuppressive agent to enhance the therapeutic effect.

Still other embodiments are directed to a method for identifying candidate target genes regulated by Mitf in B cells, comprising, i) generating a mouse model with a systemic loss-of-function mutation of Mitf or B-cell specific inactivation of the MiT family, ii) performing RNAseq analysis on resting B cells from the mouse model to identify differentially regulated genes, and iii) validating the identified genes as targets for therapeutic intervention in B cell-related autoimmune diseases.

According to an embodiment a diagnostic kit for determining the presence of autoimmune disease manifestations associated with Mitf dysfunction is disclosed herein. The kit comprises: i) probes or primers for detecting the expression levels of genes upregulated in Mitf-deficient B cells, such as Socs6, Isp53 (Baiap1), S1pR2, and IgG2b/c, ii) reagents for RNA extraction and quantification, and iii) instructions for interpreting the results.

We have discovered, inter alia, that the introduction specifically into B cells of a manufactured recombinant protein, i.e., Transdominant Negative (TDN), which is non-natural but mimics the overexpression of Tfec, causes B cells to become pathogenic and self-reactive. The excessive introduction of the Transdominant Negative (TDN) protein into B cells leads to worsened lupus-like disease via loss-of-function of Mitf and other MiT family members. Further, it is known that increased Tfec levels are observed in pathogenic B cells in autoimmune diseases, including but not limited to systemic lupus erythematosus. Thus, targeting Tfec to decrease or otherwise be inhibited, represents a rational therapeutic approach for treating autoimmune diseases characterized by increased Tfec levels in pathogenic B cells.

### Definitions

As disclosed herein, Mitf has been shown to regulate B cell activation and tolerance and is directed to the protein Microphthalmia-associated transcription factor (MITF), which plays a significant role in controlling how B cells become activated and the level of tolerance they exhibit towards self-antigens, essentially acting as a molecular switch that influences the immune response by regulating B cell function at a fundamental level; this is a relatively recent discovery that expands the known roles of MITF beyond its well-established function in melanocyte development. MITF as a regulator: MITF acts as a transcription factor, meaning it controls the expression of other genes, and in the context of B cells, it can influence genes involved in their activation and tolerance mechanisms. B cell activation: When an antigen binds to a B cell receptor, it triggers a cascade of events collectively called “activation” that includes B cell. MITF can modulate this activation process by influencing the expression of genes necessary for activation. B cell tolerance: Tolerance refers to the immune system's ability to recognize and not attack self-antigens, and MITF can help maintain this tolerance by preventing the development and function of B cells that might otherwise react to self-antigens.

As disclosed herein, MiT/TFE (microphthalmia/transcription factor E, or MiT) is a family of transcription factors that regulate a variety of cellular processes, including autophagy, lysosome biogenesis, and energy homeostasis: MiT/TFE transcription factors are made up of four genes: MITF, TFEB, TFE3, and TFEC. They share a common structure, including a basic helix-loop-helix (bHLH) leucine zipper (LZ) dimerization motif, a transactivation domain, and a DNA binding domain. Importantly, TFEC lacks an acidic domain, so when it binds to and forms heterodimers with other MiT family members, such as MITF, TFEB, and TFE3, it can inhibit their ability to activate transcription.

TDN, as disclosed herein, is a recombinant trans-dominant negative protein that is not natural and has been generated and produced in the Christopher Roman laboratory. This protein mimics the function of Tfec by heterodimerizing with Mitf, Tfe3, and Tfeb. However, it lacks a DNA binding domain, and therefore inhibits the transcription activation of MiT family members.

Mitf^{mi-vga9/mi-vga9} a germline recessive null Mitf mutation mouse strain where Mitf has loss of expression in all cell types (Also known as VGA or Mitfvga/vga). The heterozygous version of this mutation is Mitfvga/+.

A bone marrow chimera is an organism that contains a mixture of its own cells and cells from a donor, typically created by transplanting bone marrow or hematopoietic stem cells (HSCs) between different subjects. Bone marrow chimeras are a key tool in biological and immunological research, and are used to study a variety of cellular processes.

A heterodimer as disclosed herein is a complex formed by the binding of two different molecules, typically proteins, which interact to perform a biological function.

A homodimer as disclosed herein is a complex formed by the binding of two identical molecules, typically proteins, which interact to perform a biological function.

B cells, or B lymphocytes, as disclosed herein, include various subsets such as, but not limited to, double negative 2 B cells, memory B cells, regulatory B cells, aging-associated B cells, and plasmablast B cells. These subsets each have distinct roles in the immune system. The embodiments discussed herein refer to specific subsets of B cells, and/or all B cells that have the potential to become self-reactive.

Pathogenic B cells, as disclosed herein, refer to abnormal B cells that contribute to disease. This term does not imply B cells infected with a pathogen, but rather those that are dysfunctional or harmful in the context of the immune response.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 presents immunocytochemistry of TDN-B Cells. (A, B) Immunocytochemistry staining to detect total nucleated splenocytes (DAPI), TDN intracellular expression (Hemagglutinin(HA)-TDN) and B cells (B220). Representative of at least three experiments. (B) The intensity of HA staining, minus background, in B cells (DAPI and B220 stained) and non-B cells (only DAPI stained) in TDN-B splenocytes. Bars show mean with the SEM of 20-40 cells per group. (C, D) Frequencies of lymphocytes and CD19+ B cells among wildtype, TDN-B, and Mitf^{mi-vga9/mi-vga9} splenocytes. 2-4 months of age. Similar findings were observed in mice at 6 months of age (data not shown). All data shown age and sex matched, male and female mice.

FIG. 2 presents the MiT family of transcription factors (TFE3, TFEB, MITF, and TFEC) share conserved structural domains crucial for their function, as depicted in this figure. All members possess a basic region (red) for DNA binding, a helix-loop-helix domain (blue), and a leucine zipper domain (green), both contributing to dimerization. These dimerization and DNA binding domains are highlighted in the blue box. The acidic domain (pink), responsible for transcriptional activation, is present in TFE3, TFEB, and MITF, but notably absent in TFEC. The purple box highlights the ubiquitin degradation region. Potential phosphorylation sites by MAPK/Erk and p90Rsk kinases (vertical lines) are also indicated. Despite lacking an acidic domain, TFEC can heterodimerize with other MiT proteins via its helix-loop-helix and leucine zipper domains, potentially inhibiting their DNA binding and transcriptional activity.

FIG. 3 presents altered lymphoid compartments and lymphocyte subpopulations in Mitf and/or MiT/TFE deficient mice. (A, B) Spleen weight and splenocyte number relative to body weight of wildtype, TDN-B, TDN-BT, and Mitf^{mi-vga9/mi-vga9} mice. 2-4 months old. (C) Frequency of plasma cells in bone marrow (BM), spleen (Sp), and lymph nodes (LN) of wildtype, TDN-B, and Mitf^{mi-vga9/mi-vga9} mice. 3-6 months old. (D, E) Relative frequencies of multipotent germinal center precursor cells (D) and germinal center B cells (E) in wildtype, TDN-B, TDN-BT, and Mitf^{mi-vga9/mi-vga9} splenocytes. 3-6 months old. (F) Lymphocyte antigen-6A/E family protein positive B cells in wildtype, TDN-B, TDN-BT, and Mitf^{mi-vga9/mi-vga9} splenocytes. 10-12 months old. (G) Active effector (CD62LlowCD44high) helper T cell, (H) naïve helper (CD62LhighCD44low) T cell, and (I)C-C chemokine receptor type 7 helper T cell, and proportions of wildtype, TDN-B, TDN-BT, and Mitf^{mi-vga9/mi-vga9} splenocytes. 4-7 months old. Kruskal-Wallis with multiple comparisons post-hoc, error bars indicate SEM, *p<0.05. (J) Microscopy of splenic primary follicles at low magnification wide-field microscopy (top) and confocal microscopy (bottom). CD3 is shown in red, B220 is shown in green. Representative of at least three experiments; 6-8 months old. All data shown age and sex matched, male and female mice.

FIG. 4 (Provides strategy and methodology by which cell types quantified in FIG. 3. are defined) presents altered lymphoid compartments and lymphocyte subpopulations in Mitf and/or MiT/TFE deficient mice. Flow cytometry markers and gating strategies of (A) germinal center precursor B cells, (B) germinal center B cells, (C) plasma cells, (D) active effector and naïve helper T cells, (E) mature B cells, (F) follicular B cells, (G)C-C chemokine receptor type 7 helper T cells, and (H) PD-1 helper T cells. (I) MFI of CD54 (ICAM-1) and (J) proportion of CD279 (PD-1)CD4+ splenocytes from wildtype, TDN-B, TDN-BT, and Mitf^{mi-vga9/mi-vga9}. (K) Frequency of total (CD19+), mature, Follicular B cells of wildtype, TDN-B, and Mitf^{mi-vga9/mi-vga9} mice. 3-7 months old. Kruskal-Wallis w/multiple comparisons post-hoc, error bars indicate SEM, *p<0.05. All data shown age and sex matched, male and female mice.

FIG. 5 (relates to FIG. 3) presents flow cytometric evaluation of bone marrow B lymphocyte developmental stages. (A) Gating scheme of B cell development stages. (B) Frequencies of B cell stages in wildtype, TDN-B, and Mitf^{mi-vga9/mi-vga9} mice. 3-5 months old. Kruskal-Wallis w/multiple comparisons post-hoc, error bars indicate SEM, *p<0.05. All data shown age and sex matched, male and female mice.

FIG. 6 (relates to FIG. 3) presents flow cytometric evaluation of splenic B lymphocyte developmental stages. (A) Gating scheme of B cell development stages (B) frequencies of B cell subpopulations in wildtype, TDN-B, and Mitf^{mi-vga9/mi-vga9} mice. 4-6 months old. Kruskal-Wallis with multiple comparisons post-hoc, error bars indicate SEM, *p<0.05. All data shown age and sex matched, male and female mice.

FIG. 7 presents selective enhancement of antibody and autoantibody titers and proteinuria in mice with Mitf and/or MiT/TFE family deficiency. (A) Total IgG and Total IgM titers detected in peripheral blood of unimmunized wildtype, TDN-B, and Mitf^{mi-vga9/mi-vga9} mice. (left) 6-7 months old (right) 5-8 months old mice. (B) 2,4,6-Trinitrophenyl hapten-FICOLL specific (T independent) total IgG and total IgM detected in peripheral blood seven days post-immunization of wildtype, TDN-B, TDN-BT, and Mitf^{mi-vga9/mi-vga9} mice. 8-10 months old. (C) IgG ssDNA, IgG dsDNA, IgM ssDNA, and IgM dsDNA titers detected in peripheral blood of unimmunized wildtype, TDN-B, and Mitf^{mi-vga9/mi-vga9} mice. Related OD reading units OD450. WT vs. TDN-B 6-7 months old. WT vs. Mitf^{mi-vga9/mi-vga9}. 5-8 months old. (D) Serum rheumatoid factor titers in wildtype, TDN-B, TDN-BT, and Mitf^{mi-vga9/mi-vga9} peripheral blood. 5-8 months old. (E) Protein detected by colorimetric assay of urine from wildtype, TDN-B, TDN-BT, and Mitf^{mi-vga9/mi-vga9} mice. 5-8 months old. Kruskal-Wallis with multiple comparisons post-hoc, error bars indicate SEM, *p<0.05. All data shown age and sex matched, male and female mice.

FIG. 8 presents a venn diagram summary of phenotypic changes observed in TDN-B and Mitf^{mi-vga9/mi-vga9} mice. Left are abnormalities in TDN-B mice alone. Right are abnormalities in Mitf^{mi-vga9/mi-vga9} mice alone, and in the middle are shared abnormalities.

FIG. 9 presents an autoantibody production, splenomegaly, lymphadenopathy, and renal damage caused by the Ipr mutation is augmented by B cell specific MiT family inactivation. (A) Splenomegaly and (B) lymphadenopathy in Fas^{+/lpr} (control), Fas^{lpr/lpr}, and TDN-B.Fas^{lpr/lpr} mice. 4.5-7.5 months old. (C) Total IgG, IgG ssDNA, IgG dsDNA, (D) total IgM, IgM ssDNA, IgM dsDNA dsDNA titers detected in Fas^{+/lpr}, Fas^{lpr/lpr}, and TDN-B.Fas^{lpr/lpr} peripheral blood. 4.5-7.5 months old. Kruskal-Wallis w/multiple comparisons post-hoc, error bars indicate SEM, *p<0.05. 4-6 months old, Mann-whitney U, *p<0.05. All data shown age and sex matched, male and female mice.

FIG. 10 (relates to FIG. 9) presents examples of spleen and lymph nodes from Fas^{lpr/lpr}, and TDN-B.Fas^{lpr/lpr} mice. (B) Spleen and lymph node lymphocyte and CD19+ frequencies in Fas^{lpr/lpr}, and TDN-B.Fas^{lpr/lpr} mice. % lymphocyte represents % of lymphocytes gated out of total live cells and % CD19 represents percent of B cells within lymphocyte gate. (C) Anti-chromatin serum titers in Fas^{+/lpr}, Fas^{lpr/lpr}, and TDN-B.Fas^{lpr/lpr} mice. 6 months old. All data shown age and sex matched, male and female mice.

FIG. 11 presents gene expression intensities in B cells with Mitf and/or MiT/TFE family functional impairment. (A) Principal component analysis (PCA) of normalized transcripts per million data from Mitf^{mi-vga9/mi-vga9} (pink), TDN-B (green), TDN-B/T (blue), Mitf^{mi-vga9/+} (gold), and wildtype (red) B cells. (B) Percentage of transcript variance in PCA determined by PC1, 2, 3, 4, and 5. (C, D, E, F) Pairwise comparison of quantification ratios between B cells of wildtype, TDN-B, TDN-BT, Mitf^{mi-vga9/+}, Mitf^{mi-vga9/mi-vga9} B cells. Shows significantly differentially expressed transcripts, likelihood ratio q<0.05. Red transcripts indicate those quantified >+1 or <−1 with a log 2ratio. Data shown 3 months old, female mouse purified B cells.

FIG. 12 presents a pairwise comparison of transcripts with high magnitude differential expression in models of Mitf and/or MiT/TFE family functional impairment. (A, B, C, D, E, F) Scatterplots of transcripts with log 2ratio transcript expression of >1 or <−1 of Mitf and MiT/TFE family functional impairment models compared to wildtype. (A) TDN-B vs. Mitf^{mi-vga9/mi-vga9}, (B) Mitf^{mi-vga9/+} vs. Mitf^{mi-vga9/mi-vga9},(C) Mitf^{mi-vga9/+} vs. TDN-B, (D) TDN-BT vs. TDN-B, (E) TDN-BT vs. Mitf^{mi-vga9/mi-vga9}, (F) TDN-BT vs. Mitf^{mi-vga9/mi-vga9}. All transcripts are colored as follows (X,Y): Gold=(+,+), Blue=(+,−), Orange=(−,−) Green=(−,+). 3 months old. 3 months old female mouse purified B cells.

FIG. 13 presents a heatmap depicting the expression profiles of 331 differentially expressed genes across five genotypes (Mitfvga/vga, Mitfvga/+, TDN-B, TDN-B/T, and Wildtype). Each row represents a gene, and each column represents a sample, with color intensity reflecting expression level (red: high, blue: low). The dendrogram clusters samples based on expression similarity, while colored branches group genes with similar expression patterns. Text annotations highlight gene groups with distinct expression profiles across genotypes, including genes low in Mitfvga/vga (VGA) and Mitfvga/+ (HET), genes low in TDN-B only, genes high in TDN-B and VGA, and genes high in VGA and HET.

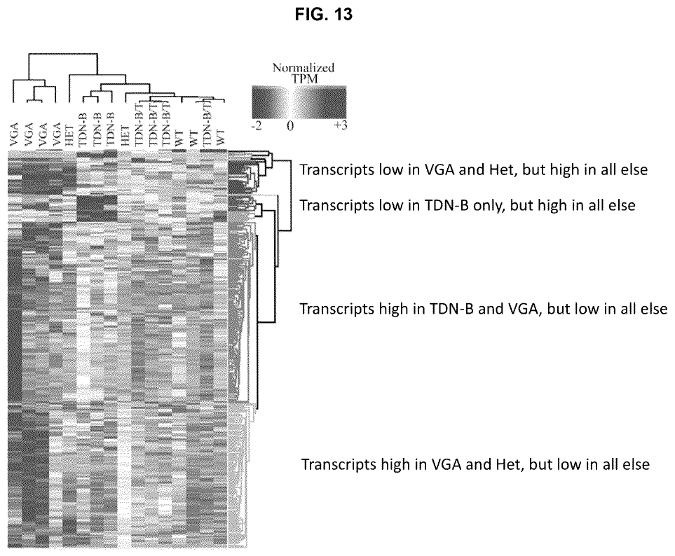

FIG. 14 presents a pathway enrichment identified through clustering of Mitf^{mi-vga9/+}, TDN-B, and Mitf^{mi-vga9/mi-vga9} transcript expression. (A) Unsupervised hierarchical clustering of significantly differentially expressed transcripts. Encircled clusters represent clusters used in pathway enrichment analysis. Clustering with average Euclidean distance linkage. Red is high, blue is low. Likelihood ratio q<0.05. (B) Pathway enrichment analysis of transcripts with increased expression in both TDN-B and Mitf^{mi-vga9/mi-vga9} B cells. (C) Pathway enrichment analysis of transcripts with increased expression in both Mitf^{mi-vga9/+} and Mitf^{mi-vga9/mi-vga9} B cells. Pathway analysis performed with Enrichr, using Reactome and KEGG annotations. Dashed line indicates Fisher's exact test qval<0.05. X axis indicates the −log(qVal) for each pathway.

FIG. 15 (relates to FIG. 14) presents the viability, proliferation, and apoptosis evaluation in B cells with Mitf and/or MiT/TFE family functional impairment. (A, B) Percent change of live (AnnexinV-Live/DeadStainlow), early apoptosis (AnnexinV+Live/DeadStainlow), and late apoptosis/dead (AnnexinV+Live/DeadStainHigh) purified B cell frequencies with and without LPS stimulation. (C) Tetrazolium (MTS) proliferation assay in wildtype, TDN-B, and Mitf^{mi-vga9/mi-vga9} purified B cells. Hours indicate time in MTS-proliferation assay post-48 hours of culture with LPS or anti-IgM+ anti-CD40 stimulation. (D) Frequencies of CD19+CD95+ in ex-vivo splenocytes of wildtype, TDN-B, Mitf^{mi-vga9/mi-vga9} mice. 3-5 months old. All data shown age and sex matched, male and female mice.

FIG. 16 presents gene expression profiling of ex-vivo B cells revealed a distinct transcriptional signature in VGA and TDN-B samples compared to HET, TDN-B/T, and WT controls. Specifically, Cluster 314, comprising 151 transcripts from 136 genes, exhibited markedly higher expression in both VGA and TDN-B. Gene ontology analysis demonstrated significant enrichment of Cluster 314 for genes involved in cell cycle processes and MHC class II antigen presentation. This upregulation was further corroborated by the expression patterns of genes within the “GOBP Cell Cycle Process” and “Reactome MHCII Antigen Presentation” pathways, respectively, highlighting a potential shared biological response in VGA and TDN-B characterized by enhanced B cell activation and proliferation.

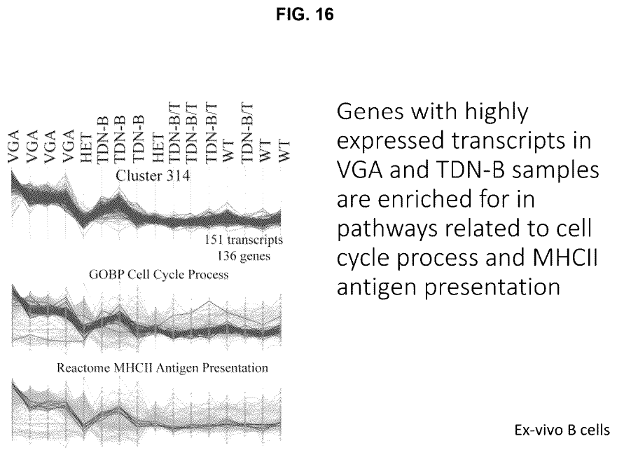

FIG. 17 presents VGA B cells have significantly higher expression of activation markers CD25 and CD69 after 24 hours of LPS stimulation in vitro, compared to stimulated wildtype B cells. Both VGA B cells and wildtype B cells were gated on CD19+ cells. Error bars represent standard error of the mean.

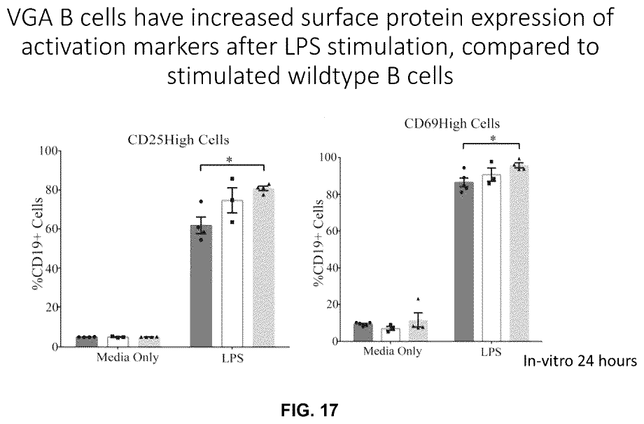

FIG. 18 VGA B cells showed increased surface expression of MHCII and CD86. In contrast, TDN-B B cells had decreased surface expression of MHCII and CD86. These findings suggest that VGA B cells have a more activated phenotype than TDN-B B cells. This difference in activation state may have implications for the function of these cells in vivo. We define here differences in antigen presentation makers on B cells with loss of MiT family members in specifically B cells (TDN-B) vs. loss of Mitf in all cells (VGA), which supports specific roles for Tfeb, Tfe3, in addition to Mitf, that regulate antigen presentation. Future therapeutics that target Tfec, will also affect the regulatory roles in B cell driven by Tfeb, and Tfe3, including of antigen presentation.

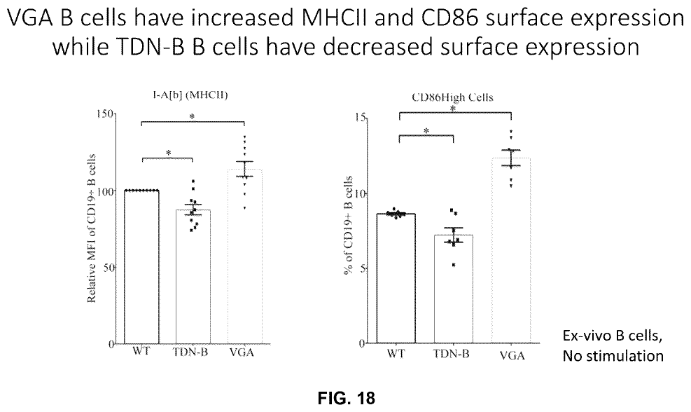

FIG. 19 presents splenic B cells from the indicated genotypes were stimulated with LPS (10 μg/mL) or left untreated for 24 hours. Cells were stained for surface MHCII and analyzed by flow cytometry. The percentage of MHCII high-expressing cells from each genotype is shown. Error bars represent the standard deviation of the mean (n=3).

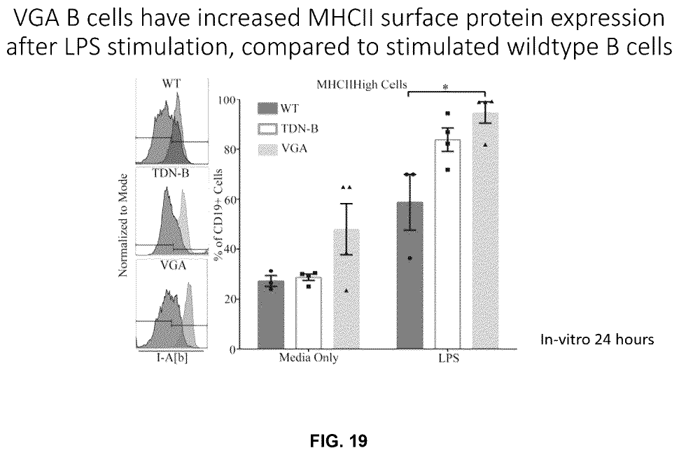

FIG. 20 presents a heatmap of differential gene expression for select transcripts in wild-type (WT), VGA, and TDN-B mice. The heatmap is colored according to z-score, with red indicating upregulation and blue indicating downregulation. Genes involved in cell proliferation, MHC antigen presentation, Toll-like receptor response, and apoptosis are shown. Notably, TDN-B mice exhibit significant upregulation of genes associated with cell proliferation and MHCII antigen presentation, while genes involved in Toll-like receptor response are downregulated.

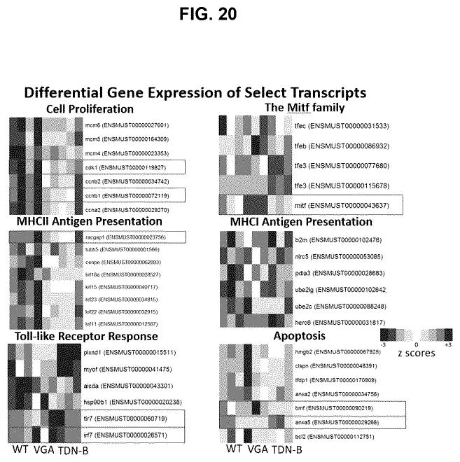

FIG. 21 presents differential expression of transcripts related to cytokine secretion and immunoglobulins with Mitf and/or MiT/TFE family functional impairment. (A) Heatmap visualization of transcripts indicated in Harris et al. 2018 to participate in innate immune signaling and type I interferon responses. *q<0.05 likelihood ratio in the present dataset. (B) Scatterplot visualization of transcripts differentially expressed within the Reactome pathway, “cytokine signaling in the immune system.” Red circles indicate genes that have >+1 in Mitf^{mi-vga9/mi-vga9} compared to wildtype B cells. 3 months old, female mouse purified B cells. (C) Concentration of TNF-alpha and IL-10 in the supernatant of purified B cell cultures after LPS and anti-IgM+ anti-CD40 stimulation. Age and sex matched, male and female 3-4 months old mouse purified B cells. Kruskal-Wallis w/multiple comparisons post-hoc, error bars indicate SEM, *p<0.05. (D) Heatmap of significantly differentially expressed transcripts from the heavy chain locus, light chain locus, and joining chain. IgG2b transcripts are encircled. 3 months old, female mouse purified B cells. (E, F, G) TNP specific IgG2c, IgG2b, and IgG1 sera levels seven days after immunization in wildtype and TDN-B mice with TNP-Ficoll. Age and sex matched, male and female 8-10 months old mice. Kruskal-Wallis w/multiple comparisons post-hoc, error bars indicate SEM, * p<0.05.

FIG. 22 presents a pathway enrichment identified through clustering of TDN-B and TDN-BT transcript expression. (A) Unsupervised hierarchical clustering of significantly differentially expressed transcripts. Encircled clusters represent clusters used in pathway enrichment analysis. Clustering with average Euclidean distance linkage. Red is high, blue is low. Likelihood ratio q<0.05. (B) Pathway enrichment analysis of transcripts with increased expression in only TDN-B, but not TDN-BT B cells. (C, D, E) Identification of select clusters from FIG. 8a. Encircled transcripts represent transcripts increased or decreased in both TDN-B and TDN-BT B cells. Pathway analysis performed with Enrichr, using Reactome and KEGG annotations. Dashed line indicates Fisher's exact test qval<0.05. #indicates two tfe3 transcripts that are increased in both TDN-B and BT due to their identity to the transdominant negative transgene.

FIG. 23 presents a venn diagram summary of gene transcription changes observed in TDN-B and Mitf^{mi-vga9/mi-vga9} mice. Left are abnormalities in TDN-B mice alone. Right are abnormalities in Mitf^{mi-vga9/mi-vga9} mice alone, and in the middle are shared abnormalities. *indicates transcript differential expression also appreciated in TDN-B/T B cells.

FIG. 24 presents a prediction analysis and consideration of theoretical direct binding sites of Mitf and/or MiT family members in B cells. (A) Scatterplot of select transcripts mapped a the log 2ratio of TDN-B/WT against Mitf^{mi-vga9/mi-vga9}/WT MiT/TFE family members are indicated in black, open circles. Blue squares and red circle are transcription factors from FIGS. 9C,D. Green diamonds indicate Mitf direct gene binding targets in mast cells, as observed through ChiP-Seq in Calero-Nieto, et al. 2014. All transcripts depicted are significantly differentially expressed, except Irf4, which is indicated by an orange circle. #Indicates that Mitf has low Mitf^{mi-vga9/mi-vga9}/WT relative expression, because Mitf has minimal to no expression in Mitf^{mi-vga9/mi-vga9} mice and that Tfe3 is indicated to have high TDN-B/WT relative expression because the transdominant negative transgene (TDN) is likely recognized as a Tfe3 transcript during RNAseq analysis. (B) Relative normalized expression of select genes, Irf4, Myc, and Mitf, with and without LPS stimulation in purified, naïve B cells. Data shown, age and gender matched male and female mouse purified B cells. 2-4 months old. (C) ChIP-X enrichment analysis 3 (ChEA3) prediction analysis of the transcript factor network related to transcripts significantly differentially expressed in B cells with Mitf and/or MiT/TFE family functional impairment. (Left) Local network visualization of 10 top-ranked transcription factors identified through ChEA3 based on the 25 transcripts in Table 1. Mef2b is encircled because it is significantly increased in TDN-B and Mitf^{mi-vga9/mi-vga9} B cells. (Right) Local network visualization of 50 top-ranked transcription factors identified through ChEA3 based on all 311 significantly differentially expressed transcripts in the present study. Encircled transcripts are transcription factors also identified as significantly differentially expressed in the present study. (Mybl1, Stat1, E2f7, Foxm1, Tfdp1, Mybl2). Additionally, two other transcription factors, Mef2b and Irf7, were identified as Top-ranked based on this gene set input by ChEA3 at ranks 58 and 81, respectively.

FIG. 25 presents the expression of MiT family members in human B cells and plasma cells. (A, B) Heatmap visualizing 72 transcripts significantly differentially expressed in B cells after Mitf and/or MiT/TFE family functional impairment also identified by Kassambara et al., 2015 to be differentially expressed in normal B cell development stages. (A) Unsupervised hierarchical clustering of 72 transcripts quantified based on significance analysis of microarrays multiclass expression z-scores access through genomicscape. (B) 72 transcripts, aligned with transcripts in FIG. 25A, quantified as log 2ratio of B cell transcripts in each Mitf and MiT/TFE functional impairment model relative to wildtype. Encircled transcripts indicate those with a similar pattern of expression in Mitf^{mi-vga9/mi-vga9} naïve B cells and human B cell subsets. 3 months old, female mouse purified B cells. (C) Mitf (D) Tfeb (E) Tfe3, and (F) Tfec Log2 GC robust multi-array average quantification identified as significantly differentially expressed across eight mature B cell stages and plasma cells. Adapted from Kassambara et al., 2015. Naïve B cells (NBCs), germinal center centroblasts (CBs), germinal center centrocytes (CCs), memory B cells (MBCs), preplasmablasts (PrePBs), early plasma cells (EPCs), plasmablasts (PBs), and bone marrow plasma cells (BMPCs).

FIG. 26 Log2 GC robust multi-array average quantification identified as significantly differentially expressed across eight mature B cell stages and plasma cells. Clockwise, Mybl2, Mef2b, Foxm1, Stat1, Socs6, E2f7, Irf4, and Mybl1. Adapted from Kassambara et al., 2015.

FIG. 27 illustrates the contrasting roles of the transcription factor Mitf in B cell biology, highlighting both its physiological importance (“Input” leading to “Output”) and the consequences of its dysregulation (“Input” leading to negative consequences). In a normal scenario (Mitf+/+), innate immune stimulation by factors like LPS or self-antigens triggers a physiological increase in surface activation markers, antigen presentation proteins, and TNF-alpha cytokine secretion by B cells. This Mitf-mediated response is crucial for maintaining B cell and T cell homeostasis, ensuring appropriate germinal center responses tailored to the encountered antigen. Conversely, excessive or dysregulated Mitf expression (Mitf−/−) driven by similar innate stimuli leads to an exaggerated response. This manifests as excessive TNF-alpha cytokine secretion, disruption of B cell and T cell organization and cross-regulation, and ultimately, an increase in germinal center B cells and plasma cells. This over-activation of the immune response can contribute to autoantibody production, highlighting the delicate balance maintained by Mitf in B cell biology.

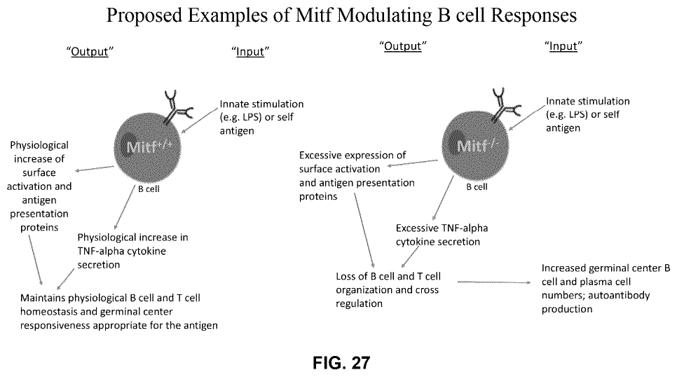

FIG. 28 presents memory B cells from SLE patients exhibit elevated TFEC expression. CD27+IgD+ memory B cells are critical for mounting effective immune responses, but dysregulation of this subset contributes to the pathogenesis of autoimmune diseases such as SLE. This figure, adapted from Moroney et al. 2020, demonstrates significantly increased expression of the transcription factor TFEC specifically within the CD27+IgD+ memory B cell population of SLE patients compared to healthy controls. This finding aligns with previous studies reporting elevated TFEC levels in pathogenic B cell subsets, including DN2 (CD27-IgD-) cells, in SLE patients (Ruschil et al. 2020, Nehar-Belaid 2021), further implicating TFEC in the aberrant B cell activity driving SLE. Tfec is also increased in age-associated B cells (ABCs)(Dai et al. 2024)

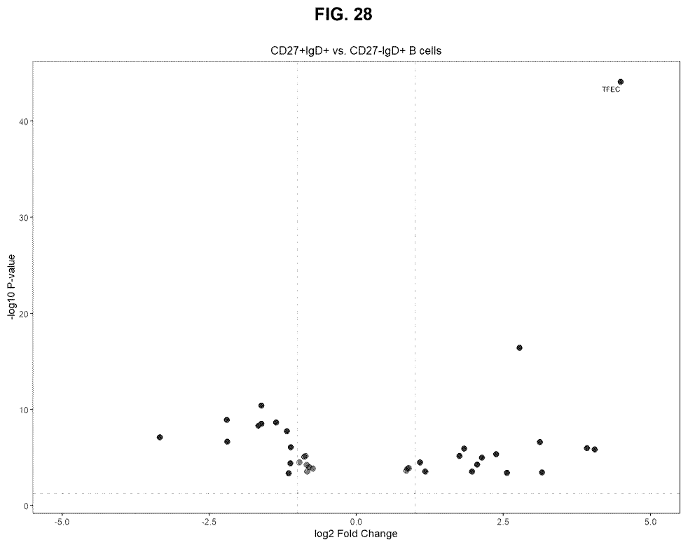

FIG. 29 presents regulatory B cells exhibit decreased expression of TFEC. The bar graph depicts the normalized expression (Z-score) of various transcription factors in regulatory B cells (adapted from Dubois et al. 2020). A negative Z-score indicates lower expression compared to other B cell subsets. Notably, TFEC (highlighted) shows significantly decreased expression in regulatory B cells. This downregulation of TFEC is consistent with the known role of TFEC to prevent the function of Mitf. With lower Tfec in regulatory B cells, Mitf is disinhibited and able to apply its regulatory roles to maintain B cell tolerance, potentially supporting anti-inflammatory and immunosuppressive functions of regulatory B cells. Error bars represent standard error of the mean.

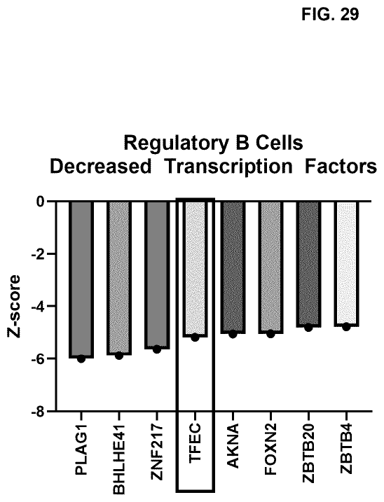

FIG. 30 presents transcriptional regulation of B Cell homeostasis by MiT family members. The MiT family of transcription factors, including Tfe3 and Tfeb, play a crucial role in maintaining B cell homeostasis. These factors bind to DNA and regulate the expression of genes involved in B cell survival, proliferation, and differentiation, particularly in response to cellular stimulants or following antigen recognition.

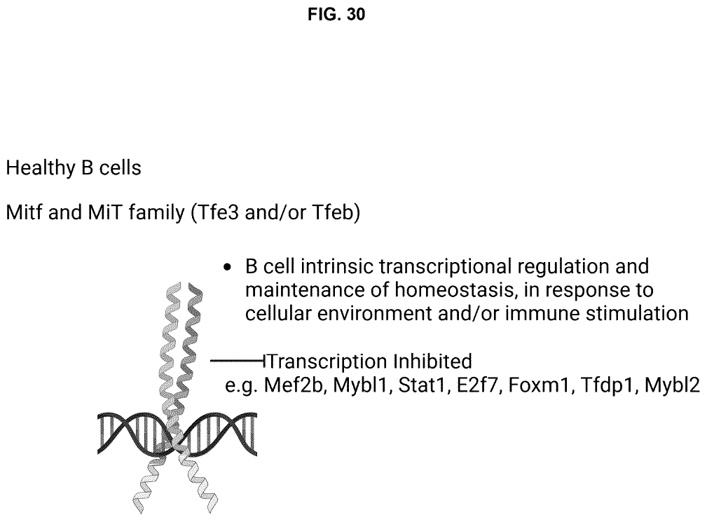

FIG. 31 illustrates the role of Mitf and the MIT family (Tfe3 and/or Tfeb) in B cell tolerance and the breakdown that leads to autoimmunity. In autoreactive B cells, Mitf and/or MIT family protein are prevented from downstream target gene functions, which includes an inability to modulate B cell homeostasis and activation. This permits the transcription of genes involved in pathogenic pathways, including Mef2b, Mybl1, Stat1, E2f7, Foxm1, Tfdp1, and Mybl2, ultimately contributing to the development of autoimmune diseases.

FIG. 32 depicts two therapeutic strategies for restoring immune tolerance in autoreactive B cells. The first strategy aims to decrease the ability of Transcription Factor E-protein (Tfec) to inactivate the transcriptional function of Microphthalmia-associated transcription factor (Mitf) and/or Tfe3/Tfeb. This can be achieved through various methods, including engineered zinc finger proteins, CRISPR-guided base editing, Transcription Activator-Like Effector Nucleases, RNA-targeted therapeutics using ADARS, small molecules targeting Tfec, and increased proteasomal degradation of Tfec. The second strategy focuses on increasing Mitf transcription and translation, potentially through engineered zinc finger proteins, CRISPR-guided base editing, and decreased proteasomal degradation of Mitf. Both strategies ultimately aim to restore the ability of Mitf (with Tfe3 and/or Tfeb) to maintain immune tolerance.

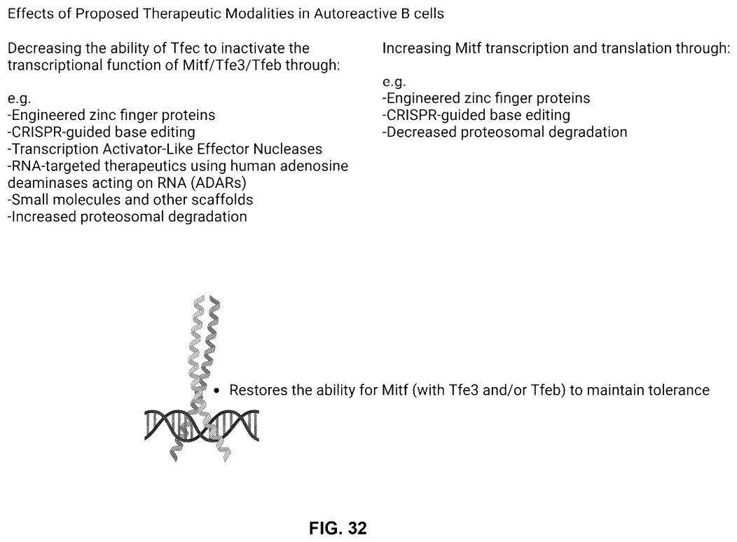

FIG. 33 presents therapeutic strategy for enhancing Mitf expression by targeting miRNA148a. This figure depicts a novel approach to increase Mitf expression by inhibiting miRNA148a, a known negative regulator of Mitf (Gonzalez-Martin et al., 2016). The therapeutic strategy involves utilizing miRNA-targeting molecules designed to specifically degrade miRNA148a. This degradation alleviates the suppressive effect of miRNA148a on Mitf, thereby promoting both transcriptional and translational upregulation of the Mitf gene. Consequently, increased levels of Mitf protein, potentially in conjunction with transcription factors like Tfe3 and/or Tfeb, can effectively execute their roles in maintaining cellular tolerance. While this figure focuses on miRNA148a, it is important to note that other miRNAs may also regulate Mitf, and this strategy could be broadly applicable to those miRNAs as well.

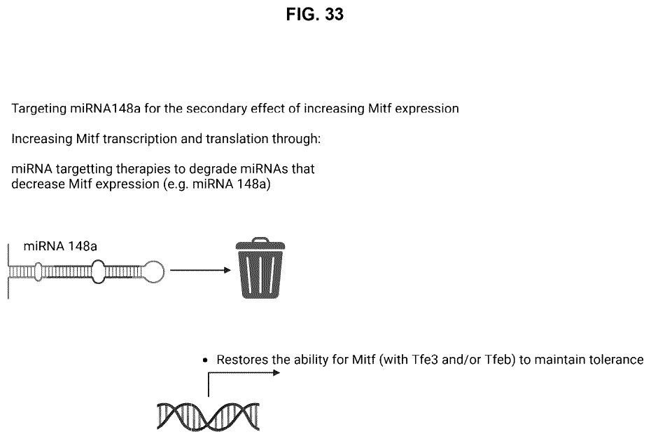

### DETAILED DESCRIPTION OF THE INVENTION

The microphthalmia transcription factor Mitf has been shown to regulate B cell activation and tolerance. However, the underlying B cell-specific mechanisms responsible, and those that distinguish Mitf from closely related Mitf/TFE (MiT) transcription factors Tfe3, Tfeb, and Tfec, remain obscure. Two complementary mouse models of Mitf and MiT deficiency were used to define a model of MiT family candidate target genes and pathway dysregulation that can occur in pathogenic B cells due to Tfec-like overexpression and subsequent loss-of-protective functions of Mitf for autoimmune tolerance. We utilize the Mitf^{mi-vga9/mi-vga9} systemic loss-of-function mutation mouse model, and introduce a transgenic, non-natural, trans-dominant negative (TDN) protein into B cells (called TDN-B) to model B-cell specific MiT family interactions that, similar to Tfec, inactivates transcription of MiT family members, including Mitf. Both models displayed spontaneous splenomegaly coincident with elevated plasma cell numbers, autoantibody titers, and proteinuria. These abnormalities appeared dependent on T helper cells, but independent of other non-B cell intrinsic effects of systemic Mitf inactivation. MiT inactivation in B cells augmented aspects of lupus-like autoimmune disease on the C57BL/6-Fas^{lpr/lpr} background. In both models, RNAseq of ex vivo resting B cells showed transcriptional upregulation of genes that control cell cycle, germinal center responses, and plasma cell differentiation. Among the genes strongly upregulated in both models were Socs6, Isp53 (Baiap1), S1pR2, and IgG2b/c. Mitf null B cells, but not TDN-B cells, showed evidence of type I interferon dysregulation implicating B-lymphocyte non-autonomous mechanisms caused by systemic absence of Mitf. With the introduction of the recombinant, TDN in B cells, we clarify Mitf's role as 1) a key regulator of a B cell intrinsic germinal center program that influences self-tolerance through novel target genes, and 2) a regulator of systemic inflammatory processes that can impact the B cell microenvironment. These findings underscore the critical role of Mitf in maintaining B cell homeostasis and self-tolerance, and highlight the potential for future therapeutics to either decrease the functionality of Tfec and/or increase the functionality of Mitf to treat autoimmune diseases.

Microphthalmia-associated transcription factor (MITF), has been shown to play a role in mature B cell homeostasis and tolerance. Lin et al., (2005) showed that bone marrow chimeras, in which the grafted hematopoietic stem cells (HSCs) harbored a dominant negative mutation of Mitf (Mitf^{mi/mi}), developed a lymphoid compartment in which a higher frequency of B cells was found to spontaneously differentiate into plasma cells in naïve mice. Also, these chimeric mice produced elevated levels of autoantibodies such as rheumatoid factor (RF) and anti-ssDNA(1). Complementary evidence from isolated Mitf^{mi/mi} B cells in culture supported the conclusion that Mitf acted to restrain B cell activation by repression of Irf4, a key regulator of plasma cell development, given that in Mitf's functional absence, Irf4 levels were elevated.

However, given that the Mitf^{mi/mi} HSC transplantation model required a full reconstitution of all bone marrow derived cells by adoptive transfer into Rag-2-deficient mice, it remains unknown whether B cell specific loss of MiT family function leads to similar abnormalities. Furthermore, since the Mitf^{mi} mutation encodes for a protein that lacks DNA binding ability but still retains the ability to dimerize with all co-expressed MiT family members, dominant negative effects may contribute to those abnormalities. Thus, it is important to evaluate the effects of loss of Mitf alone and compare that to the situation where all MiT family function is blocked.

To address this, we compared the B cell compartments of two complementary mouse models for Mitf inactivation: a single gene model for B cell-specific MiT family inactivation, called TDN-B, in which a recombinant dominant negative inhibitor of the MiT family is expressed in B cells only, and a germline recessive null Mitf mutation, Mitf^{mi-vga9/mi-vga9} that cannot inhibit other MiT family proteins. With this strategy, we assessed whether TDN-B and Mitf^{mi-vga9/mi-vga9} mice presented with similar B cell abnormalities observed in the Mitf^{mi/mi} HSC transplantation (HSCT) model. To gain further insight into how the loss of function of MiT molecules impacted control of B cell homeostasis and autoimmunity, we crossed the TDN-B gene onto the C57BL/6-Fas^{lpr/lpr} background and found enhancement of a subset of lupus-like autoimmune disease manifestations. A separate line of TDN transgenic mice, in which the TDN was expressed in both B and T cells (TDN-B/T) and has impaired T cell function as a result, was used to resolve B cell intrinsic abnormalities caused by Mitf inactivation from those dependent on T cell function. RNAseq analysis was performed on resting B cells from the different MiT/Mitf impaired strains to identify differentially regulated genes that could reveal underlying mechanisms that might explain the common and unique B cell abnormalities in each model. Our results show that inactivation of Mitf and the MiT family can impair B cell tolerance and perturb homeostasis mechanisms by derepressing a gene transcription program that regulates activation and germinal center responses.

We further hypothesize that the TDN in this context is mimicking the activity of MiT family member Tfec, which has been shown to inhibit the activity of Mitf, Tfe3, and/or Tfeb. Many studies have shown that Tfec expression is elevated in pathogenic B cells in lupus. Therefore, a reasonable therapeutic goal would be to target Tfec for inactivation based on the hypothesis that Tfec, like TDN, is inhibiting Mitf activity and thereby impairing tolerance, leading to lupus-like autoimmunity and disease.

The microphthalmia transcription factor, i.e., Mitf, has been shown to regulate B cell activation and tolerance. However, the underlying B cell-specific mechanisms responsible, and those that distinguish Mitf from closely related Mitf/TFE (MiT) transcription factors Tfe3, Tfeb, and Tfec, remain obscure. Two complementary mouse models of Mitf and MiT deficiency—the Mitfmi-vga9/mi-vga9 systemic loss-of-function mutation, and B-ell specific MiT family inactivation via transgenic expression of a trans-dominant negative (TDN) protein (TDN—B)—were used to identify MiT family candidate target genes and pathways. Both models displayed spontaneous splenomegaly coincident with elevated plasma cell numbers, autoantibody titers, and proteinuria. These abnormalities appeared dependent on T helper cells, but independent of other non-B cell intrinsic effects of systemic Mitf inactivation. MiT inactivation in B cells augmented aspects of lupus-like autoimmune disease on the C57BL/6-Faslpr/lpr background. In both models, RNAseq of ex vivo resting B cells showed transcriptional upregulation of genes that control cell cycle, germinal center responses, and plasma cell differentiation. Among the genes strongly upregulated in both models were Socs6, Isp53 (Baiap1), S1 pR2, and IgG2b/c. Mitf null B cells, but not TDN-B cells, showed evidence of type I interferon dysregulation implicating B-lymphocyte non-autonomous mechanisms caused by systemic absence of Mitf. These studies clarify Mitf's role as 1) a key regulator of a B cell intrinsic germinal center program that influences self-tolerance through novel target genes, and 2) a regulator of systemic inflammatory processes that can impact the B cell microenvironment.

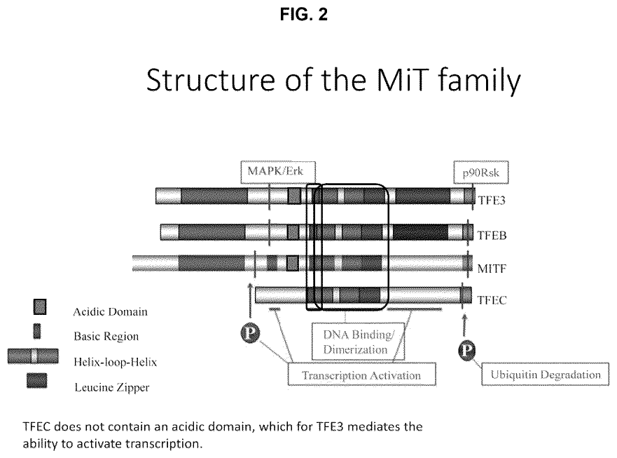

Material and Methods: All mice were housed in specific pathogen-free conditions in the Division of Comparative Medicine at the State University of New York-Downstate Health Sciences University (SUNY Downstate); all experimental procedures were conducted according to protocols approved by its Institutional Animal Care and Use Committee. A recombinant gene encoding the trans dominant negative (TDN) protein was constructed from the TFE3 helix-loop-helix leucine zipper dimerization domain that lacks the DNA binding basic region, but was fused to a nuclear localization sequence and a hemagglutinin epitope. The TDN-transgene contained the TDN cDNA subcloned into an immunoglobulin heavy-chain gene enhancer and promoter-based transgene cassette (EμPϕ that directs expression in B cells (TDN-B) or both B and T cells (TDN-B/T) (FIG. 2). Mitf^{mi-vga9/mi-vga9} and Mitf^{mi-vga9/+} mice were a kind gift of the Arnheiter lab, and C57B/6-Fas^{lpr/lpr} were obtained from The Jackson Laboratory. To generate TDN-B.Fas^{lpr/lpr} mice, TDN-B animals (>9 generations backcrossed TDN-B onto C57BL/6) were intercrossed with C57B/6-Fas^{lpr/lpr} animals. The presence of the TDN transgene was detected by PCR on tail or ear DNA using primers 5′-CACATAACAACCACATTCCTCCT-3′ and 5′-GAGCGTCCACAGGACCCTGAG-3′. Mitf^{mi-vga9/mi-vga9} mice were identified by their albino coat color. Mitf^{mi-vga9/+} were non-albino siblings of albino mice from litters of crosses between Mitf^{mi-vga9/+} females and Mitf^{mi-vga9/mi-vga9} males and spot checked by PCR detection of the insertion transgene. All TDN transgenic mice used for experiments were hemizygous for the TDN transgene; control mice were non-transgenic, sex-matched littermates of TDN-transgenic mice.

B cell enrichment: For RNA sequencing and in vitro culture studies, mouse B cells were enriched from splenocytes of 3-month-old, female mice using the Mouse B Cell EasySep Isolation Kit (StemCell Technologies, BC, Canada). Specifically, B cells were purified by negative selection against CD43, CD49b, CD90. 2, GR-1, TER119, CD11b, CD8a, and CD4, and including an FcR blocking antibody recognizing mouse CD16/CD32, to a purity of 96% or more as determined by flow cytometry. Purified B cells were counted by a TC-20 automated cell counter (Bio-Rad).

Flow cytometry and detection antibodies: For detection of cell surface markers, single-cell suspensions of splenocytes or bone marrow cells were treated with red blood cell (RBC) lysis buffer to eliminate erythrocytes and then stained following standard protocols. The following antibodies to characterize and enumerate B cells were used: CD19, CD21, CD23, IgM, IgD, CD69, CD25, CD95, CD38, CD138, Ly6A/E, Ly6C, AnnexinV, I-A[b], and CD86. Markers for T cell studies were: CD4, CCR7, CD5, CD54, CD279, CD62L, and CD44. Antibodies were conjugated to FITC-, PE-, PerCP-Cy5.5-, PE-Cy7-, orAPC-Cy7, and were purchased from eBiosciences or BD Biosciences. Data acquisition was performed on a FACScan (BD Biosciences) or Guava easyCyte flow cytometer (EMD Millipore) and analyzed by CellQuest or FlowJo software (v10.0.07). Live vs. dead cells were distinguished on the Guava EasyCyte using the Live/Dead Fixable Far Red Dead Cell Stain kit (ThermoFisher Scientific). Lymphocyte gate was determined by forward scatter vs. side scatter properties.

Immunizations: To evaluate thymus-independent (TI) responses to a model type II antigen, ˜8-month-old wild-type, TDN-B and TDN-B/T mice were immunized intraperitoneally with 20 μg of the trinitrophenol (TNP) conjugated to Ficoll (Biosearch Technologies) in 1× PBS. Mice were bled right before and 7 d after immunization.

Enzyme-linked immunosorbent assay (ELISA) studies were completed to measure total, autoimmune, and TNP-specific antibodies. In brief, 96-well plates were bound by anti-IgM or anti-IgG antibodies at 10ug/mL, at room temperature (RT) incubation for 1 hour. Plates were washed with 1× TBS-Tween20 (TBS-T) 5 times. Blocking buffer was then added (1% BSA in 1× TBS) to each well to block any non-specific binding sites on the surface and incubated for one hour at RT Sera were diluted for IgM at 1:10,000 and for IgG at 1:50,000. Plates were then washed 5 times with TBS-T. HRP antibody against either IgM or IgG was diluted with blocking buffer followed by a series of dilutions, followed by incubation for one hour. Plates were washed again with 1× TBST. TMB was then added to develop positive signal (blue color). The color reaction was stopped with a 1:10 dilution of 1M H2SO4 and read immediately in a plate reader at 450 nm.

For autoantibody assays, serum levels of IgM and IgG anti-ssDNA and anti-dsDNA were detected by incubating serum diluted 1:100 into poly-L-lysine plates precoated with either ssDNA or dsDNA. Detection antibodies were IgM-HRP and IgG-HRP. Serum anti-chromatin antibodies were detected with chicken chromatin.

TNP titers were measured in Immuron plates (Dynex) coated with TNP-bovine serum albumin (BSA) (Biosearch Technologies) using HRP conjugated ELISA detection antibodies (Bethyl). Plates were incubated with 10 μg/mL of coating antibody (Bethel Laboratories). Sera were diluted in blocking buffer at 1:10,000 for IgM and 1:10,000 for IgG. For IgG isotypes, the same protocol was followed with a variation in the concentration of the isotypes: 1:40,000 for IgG2b and IgG2c.

B cell culture and supernatant multiplex assays: Purified B cells were cultured in RPMI medium (Hyclone) supplemented with 10% heat-inactivated FBS (Corning), 1% L-Glutamate (w/v), 1% Streptomycin and Ampicillin (w/v), and 55 μm 3-mercaptoethanol (Sigma), at a density of 2-2.5 million cells/mL. For stimulation, purified mouse splenic B cells were cultured with lipopolysaccharide (25 μM/mL, Salmonella enterica serotype typhimurium) or IgM (10 μg/mL) and CD40L (1 μg/mL) for 20-24 hrs. The abundance of cytokines in culture supernatants was measured using the Mouse Cytokine/Chemokine MILLIPLEX MAP multiplex kit (EMD Millipore, Burlington, MA) with a dual laser, flow-based multiplex assay (Luminex 200 TM, Luminex Corporation, Austin, TX) allowing simultaneous quantification of IL-10 and tumor necrosis factor-alpha in single samples. All sample assays were carried out in triplicate and averaged to generate a value for each biological replicate.

RNA-Sequencing and Analysis: For RNA-sequencing (RNA-seq) of highly enriched ex-vivo B cells, between 1 and 5 million cells were disrupted in Trizol Reagent and samples shipped on ice overnight to the laboratory of ES at the University of Iceland Biomedical Center. Isolation of mRNA from total RNA was accomplished with magnetic beads conjugated to oligo dT; then, RNA fragmentation, random priming, cDNA synthesis, adaptor ligation, and PCR enrichment was completed, using the NEBNext Ultra Directional RNA Library Prep Kit for Illumina. A bioanalyzer (Agilent), was used as an RNA-seq library preparation quality check. 30 million reads per sample, with a read length of ˜2×125 bp, were sequenced by paired end sequencing, and then aligned to the Mus musculus transcriptome by Kallisto, utilizing over 45,000 transcripts evaluated that passed filtering quality control in Sleuth. In each of the samples analyzed, expression was normalized as transcript per million. Gene expression results were displayed in the Perseus platform. Differential gene expression analysis was calculated with Sleuth in R. Significance was defined by q value <0.05 based on the likelihood ratio test for comparison of transcript abundance among models. The likelihood ratio does not produce a metric equivalent to the fold change, rather, the Wald test is used to define differential expression between each sample pairing. The Wald test beta statistic approximates the log 2 fold change in expression between two conditions tested. The likelihood ratio is considered the statistical test with fewer false positives compared to the Wald test, thus, significance filtering is based on likelihood q values. Beta values (natural log 2 with technical variation removed) was converted to log 2 fold change using the formula LOG(POWER(2.71828, (Beta Value)), 2) in Excel. Pathway enrichment analysis was completed through Enrichr. ChEA3(12) was utilized for transcription factor network analysis with results output as “Top Rank,” indicating the top integrated rank across libraries.

Real time RT-PCR: Total RNA from approximately 9 million purified splenic B cells per time point were analyzed via real time RT-PCR. RNA was isolated with Trizol Reagent (Ambion) and Direct-zol RNA mini-prep plus kit (Zymo) according to the manufacturer's instructions. Total RNA was reverse-transcribed by Verso cDNA synthesis Kit (Thermo Scientific) according to the manufacturer's instructions with a 3:1 blend of random hexamers and anchored oligo-dT primers for first-strand synthesis. cDNA was amplified by real time-PCR with the Maxima SYBR Green qPCR Master Mix (Thermo Scientific) in a CFX384 Touch Real-Time PCR Detection System (Bio-Rad). All RT-qPCR experiments were completed in triplicate. The relative abundance of mouse cDNA expression was measured relative to P-Actin. PCR analysis Data Collection, Data Analysis, and Statistics were completed with the CFX Maestro Software (Bio-Rad). All primers used had an efficiency greater than 90% and a delta-delta Ct was used to assess relative qPCR amplification between samples.

Immunostaining: Spleen sections were snap-frozen embedded in OCT (Tissue Tek) and stored at −80C until sectioning. 14 μM thick sections on slides were fixed in Acetone for 10 minutes. Slides were then removed from the fixation agent and dried for 10 min, and then washed three times with PBS+0.1% Tween-20 (PBS-T) and incubated for 1 hr with blocking solution containing 5% normal goat serum and 0.3% Triton x-100 diluted in PBS-T. After the blocking solution was removed, the primary antibody was applied to the tissue section overnight at 4° C., in a humid chamber, at the following dilutions: anti-B220 (primary 1:250 dilution, abcam, RA3-6B2), anti-CD3 (1:200, abcam, ab5690). After three PBS-T washes, tissue sections were then incubated with secondary antibody for 2 h at room temperature, at the following dilutions: anti-rat Alexa488 (1:100, abcam, ab150165), anti-rabbit Alexa594 (1:200, abcam, ab150084). Sections were again washed three times in PBS-T and then either incubated with counterstain, or next mounted with Hardset Antifade Mounting Medium (Vecta Shield) and covered with a coverslip. Sections were viewed and imaged by wide-field, compound fluorescence microscopy (Axio Vision 4.7, Carl Zeiss), and/or confocal microscopy (Leica TCS SP-5). Immunoreactivity was measured using FIJI ImageJ software program. For fluorescence staining, splenocytes were smeared onto a poly-L-lysine coated slide (Fisherbrand, Plus), fixed with 4% Formaldehyde for 15 minutes at room temperature, and next fixed with 100% ice-cold methanol for 10 minutes in a −20C freezer. Blocking, staining, washing, and mounting were completed as indicated above.

Statistical analyses: Kruskal-Wallis test was used with multiple comparisons post-hoc to analyze flow cytometry and other biometric data. Error bars in figures are standard error of the mean. Experiments were completed with n >3 biological replicates per group. For RNA sequencing analyses, after Kallisto alignment and filtering, normalized data was completed through Sleuth. Significant differential expression was determined through the likelihood goodness-of-fit model comparing wild type gene expression (null model) against the Mitf and MiT family functional impairment sample gene expression (alternative model) at a threshold of q-value (adjusted p-value) <0.05. For pathway enrichment, significantly enriched pathways were assessed through Fisher's exact test at an adjusted p value (qval) cut off <0.05. Data analysis was graphed with Prism 7 or 8 (GraphPad) software or Perseus. Meta-analysis of microarray data from a prior study of human B cells utilizes significance analysis of microarray (SAM) multiclass analysis, unpaired Wilcoxon statistics, with a fold change>2 to define significance of differential expression.

Results: Systemic (germline) inactivation of Mitf or B-cell-specific MiT lead to spontaneous abnormalities of the mature B cell compartment. Original studies revealing a role of Mitf in regulating B cell function used a bone marrow chimera model in which HSCs from the Mitf^{mi/mi} mouse strain were engrafted to Rag-2 deficient recipients. This strategy was necessitated because of the severe osteopetrosis caused by the Mitf^{mi} mutation, which leads to lethality 3 weeks after birth. The Mitf^{mi} mutant protein is dominant negative and inactivates Tfe3, which acts redundantly with Mitf during osteoclastogenesis. In the engrafted chimeras, a higher frequency of Mitf^{mi/mi} B cells were found to spontaneously differentiate into plasma cells. However, given that the entire hematopoietic system was derived from Mitf^{mi/mi} mice, it was unclear whether a loss of MiT function only in B cells would yield a similar phenotype. Myeloid lineage cells and dendritic cells are known to influence B lymphocytes, and their properties in this capacity could have been influenced by the dominant negative Mitf mutation. Similarly, given the dominant negative property of the mutant Mitf protein produced by the Mitf^{mi} allele, it was unclear whether a strict loss-of-function Mitf mutation by itself would yield a similar B cell phenotype.

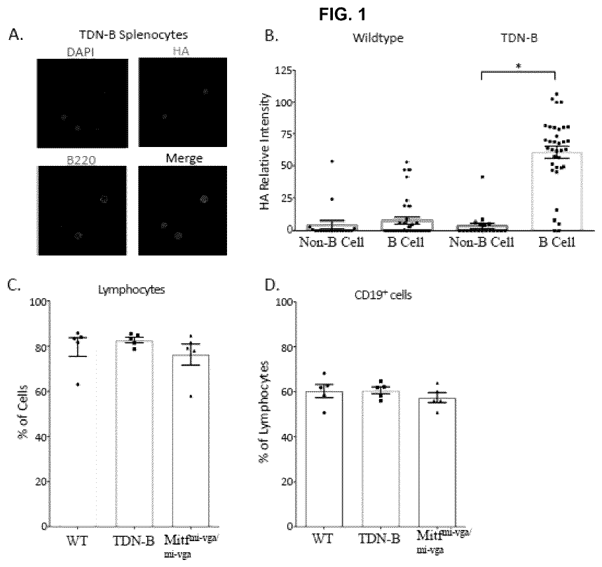

We utilized a previously described transgenic approach to express a recombinant trans dominant negative (TDN) protein specific for the MiT family in lymphocytes. The TDN protein contains the Tfe3 helix-loop-helix-leucine zipper dimerization domains, but lacks the DNA-binding basic region and transcription activation domains. This property, along with engineered nuclear localization sequences, allows it to form inactive heterodimers with endogenous MiT proteins that are incapable of binding DNA. We first focused on one line, TDN-B, in which the TDN is expressed exclusively in the B lineage (FIG. 1), as a model for B-cell specific MiT inactivation. In parallel, we evaluated a mouse homozygous for the recessive loss-of-function (“null”) allele Mitf^{mi-vga9}. These mice have a white coat and severe microphthalmia due to the requirement of Mitf for the development of both melanocytes and retinal pigment epithelial cells. However, unlike Mitf^{mi/mi}, these mice do not have osteopetrosis because Tfe3, redundantly required for osteoclast development, is not inhibited.

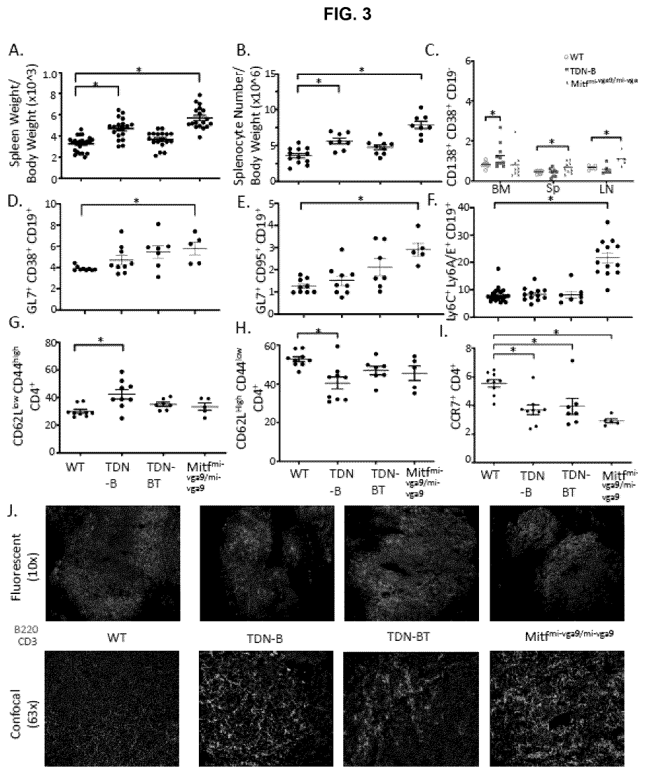

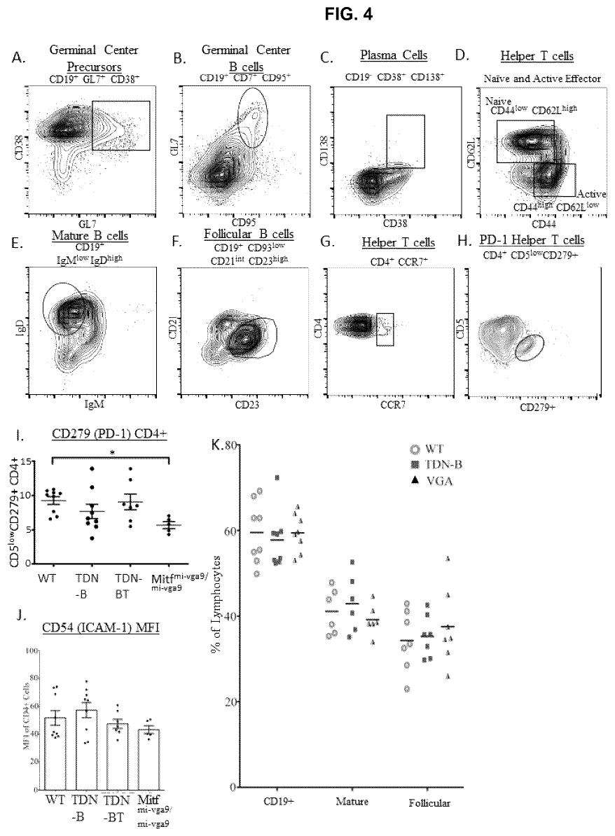

Both TDN-B and Mitfmi-vga9/mi-vga9 mice exhibited spontaneous development of splenomegaly, evident starting at 3 months (FIG. 3A), which correlated with a roughly proportional increase in total splenocyte numbers (FIG. 3B). Within this expanded population, the relative proportion of CD19+ cells appear unchanged (FIG. 1). In addition, B cell developmental stages in the bone marrow and spleen were largely intact, with a subtle increase in the proportion of pre-B cells and decrease in the proportion of marginal zone B cells noted in TDN-B mice (FIGS. 4, 5, 6). However, flow cytometry showed that the percentages of cells with markers consistent with antibody secreting plasma cells (CD19-CD38+CD138+) were elevated in both TDN-B and Mitfmi-vga9/mi-vga9 mice when compared to controls (FIG. 3C). This was most evident in Mitf^{mi-vga9/mi-vga9} spleens and lymph nodes, and the bone marrow of TDN-B mice (FIG. 3C). Multipotent germinal center precursor B cells (CD19+GL7+CD38+) and germinal center B cells (CD19+CD95+GL7+) were significantly increased in Mitf^{mivga9/mi-vga9} mice spleens (FIG. 3D, and FIG. 3E).

Recent studies on the consequences of Mitf haplo-insufficiency on melanocyte progenitor cells have suggested that global Mitf deficiency dysregulates innate immune responses systemically, leading to elevated expression of type I interferon (IFN) genes. Elevated type I IFN is also a characteristic of lupus autoimmunity. To determine whether the B cell abnormalities observed in both models were a reflection of that generalized defect, we analyzed lymphocytes for expression of markers known to be upregulated by type I IFNs. Interestingly, B cells from Mitf^{mi-vga9/mivga9} mice had an increased frequency of CD19+ B cells expressing surface Ly6C and Ly6A/E compared to controls (FIG. 1F). However, TDN-B transgenic B cells were indistinguishable from non-transgenic controls and showed no evidence of this change. We therefore conclude that ostensibly resting B cells from Mitf^{mi-vga9/mi-vga9} mice indeed express markers consistent with a deregulation of type I IFN responsive genes, and that these abnormalities do not contribute to splenic B cell compartment abnormalities in TDN-B mice. Thus, the impact of Mitf inactivation on B cells is distinct from the generalized systemic defect in type I IFN homeostasis.

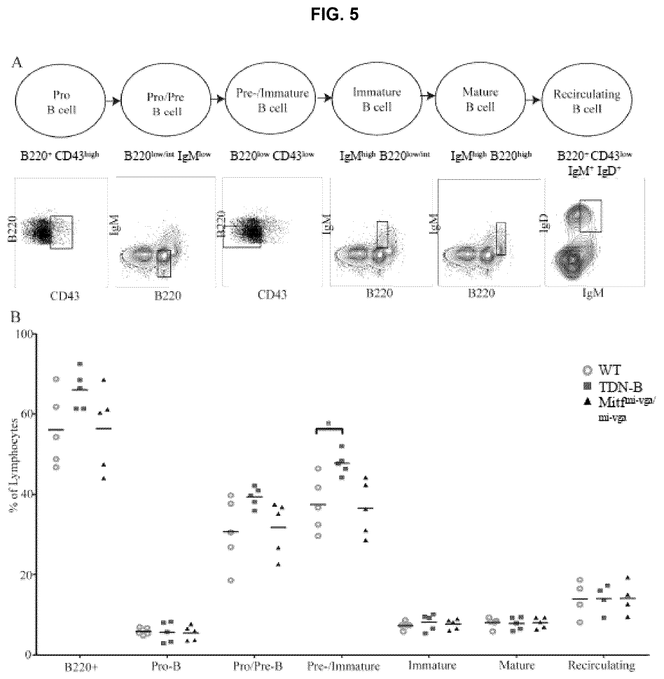

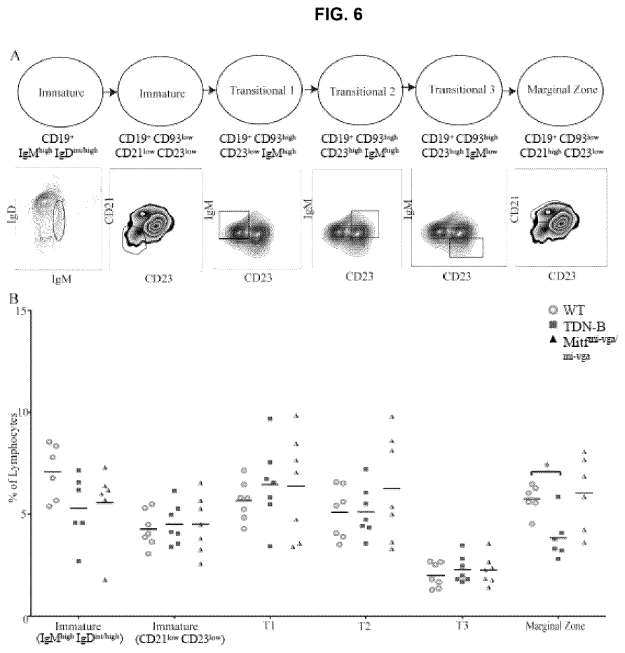

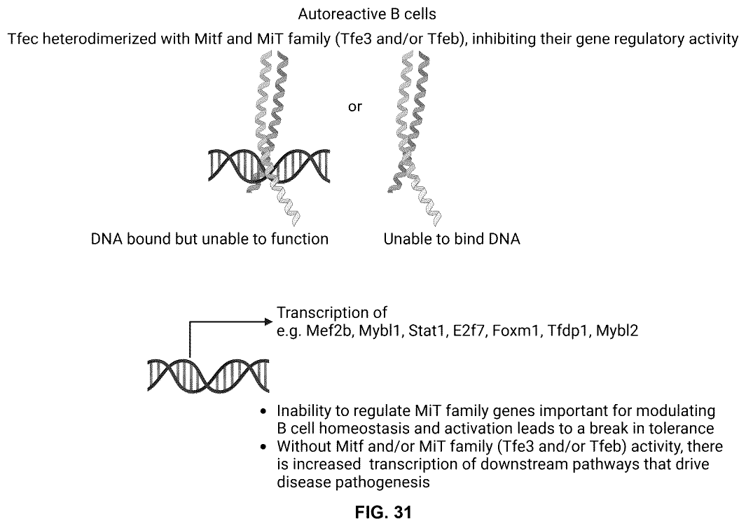

Coincident with splenomegaly, spleens from naïve TDN-B and Mitf^{mi-vga9/mivga9} mice also contained many follicles that appeared disorganized, characterized by the lack of discrete delineation between the peri-arteriolar lymphoid sheath and follicles, with confocal microscopy showing CD3+ cells within B cell (B220+) areas (FIG. 3J). This led us to analyze splenic CD4+ T cells by flow cytometry. This revealed some subtle differences in the Mitf-null and MiT-inactivated strains, with differences from control mice not always being identical to each other. For example, in TDN-B, but not Mitf^{mi-vga9/mi-vga9} spleens, the fraction of CD62LlowCD44hi CD4+ cells, indicative of effector helper T cells, was elevated (FIGS. 3G, 3H, and FIG. 2D), whereas in all Mitf-deficient models (null and inactivated) the fraction of CD4+ cells expressing the homing molecule CCR7 was decreased (FIG. 31). In contrast, Mitf^{mi-vga9/mi-vga9} spleens had a reduced frequency of CD279(PD-1)+CD4+ T cells, with TDN-B spleens trending similarly (FIG. 4). The frequency of CD54(ICAM-1)+CD4+ T cells was not different among groups (FIG. 4). There were no significant changes in B cell compartments for immature, mature, recirculating, or T1, T2, and T3 B cells (FIGS. 5 and 6). These data indicate that CD4+ T cells were responding to changes caused by the inactivation of MiT family members in B cells, but also were affected by non-B cell autonomous functions driven by unknown effects of Mitf loss-of-function or MiT inactivation in non-B cells in each model.

Spontaneous B cell abnormalities in TDN-B mice are influenced by helper T cells: To explore whether T-cell help is required for the B cell abnormalities in TDN-B mice, we analyzed a different TDN line, TDN-B/T, in which the TDN transgene was expressed in both B and T cells. As we have reported, TDN expression exclusively in T cells impairs helper CD4 T cell function by reducing expression of CD40L, which is regulated jointly by Tfe3 and Tfeb. In the TDN-B/T model, splenocyte cellularity and mass (FIGS. 3A, 3B), and its follicular architecture (FIG. 3J), were virtually indistinguishable from controls. Of note, the splenic compartment in TDN-B/T mice still contained significantly decreased CCR7+CD4+ T cells similar to TDN-B and Mitfmi-vga9/mi-vga9 spleens (FIG. 31). Overall, these observations suggest that B cell intrinsic drivers are responsible for splenic B and T cell subset changes.

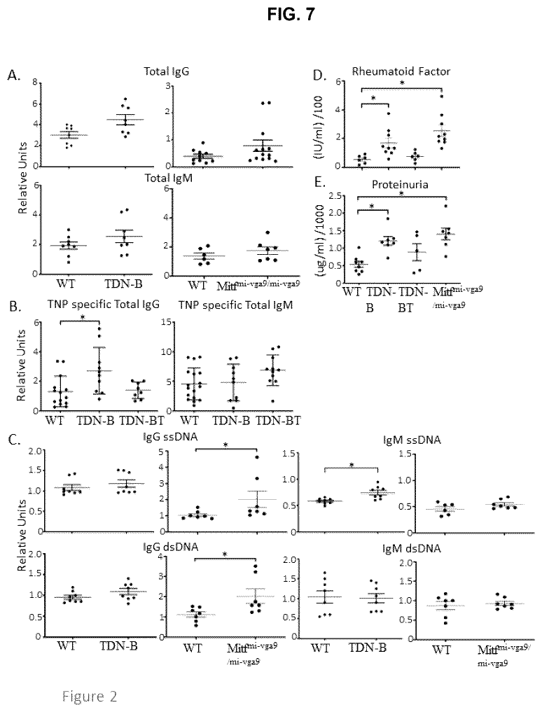

MiT family inactivation in B cells selectively influences spontaneous and induced antibody titers: Given the spontaneous B cell abnormalities in TDN-B and Mitf^{mi-vga9/mivga9} mice, we measured serum antibody levels. Since serum from unimmunized TDN-B and Mitf^{mi-vga9/mi-vga9} mice showed no significant difference in IgG or IgM levels compared to controls (FIG. 7A), we measured levels of induced antigen-specific antibody produced because of immunization. Given the possible confounding effects of Mitf-deficiency on other cells that are involved in vaccine responses, we focused the assessment on TDN-B mice, and immunized them using a standard thymus-independent (TNP-Ficoll) vaccine protocol. At day 7 post-TNP-Ficoll immunization, anti-TNP IgM in serum from TDN-B mice was the same as controls and TDN-B/T mice, but total anti-TNP IgG levels were elevated in TDN-B mice compared to controls and TDN-B/T mice (FIG. 7B). Therefore, the helper T cell-dependent changes due to Mitf and MiT family dysfunction include a modest and selective enhancement of induced and spontaneous antibody production.

MiT inactivation augments aspects of lupus-like disease in Fas^{lpr/lpr} mice: Mitf^{mi/mi} HSC chimeric mice were reported to have elevated levels of anti-ssDNA and RF. Analysis of TDN-B and Mitf^{mi-vga9/mi-vga9} sera showed similarly modest elevations of these autoantibody titers (FIGS. 7C, and 7D). Also consistent with the HSCT model, both strains had elevated titers of rheumatoid factor (FIG. 7D), which appeared to be helper T cell dependent, as this was not evident in the TDN-B/T strain. Like the HSCT model, 6-8 month old TDN-B mice had elevated levels of anti-ssDNA IgM, whereas Mitf^{mi-vga9/mi-vga9} sera contained elevated anti-dsDNA IgG (FIG. 7C). Both TDN-B and Mitf^{mi-vga9/mi-vga9} mice had increased proteinuria compared to WT and B/T mice (FIG. 7E), which correlated with rheumatoid factor levels (FIG. 7D). Thus, the type of elevated autoantibody production was similar in both strains and paralleled what was observed in the Lin et al(1). study. FIG. 6 summarizes phenotypes and B cell abnormalities in TDN-B and Mitfmi-vga9/mi-vga9 mice.

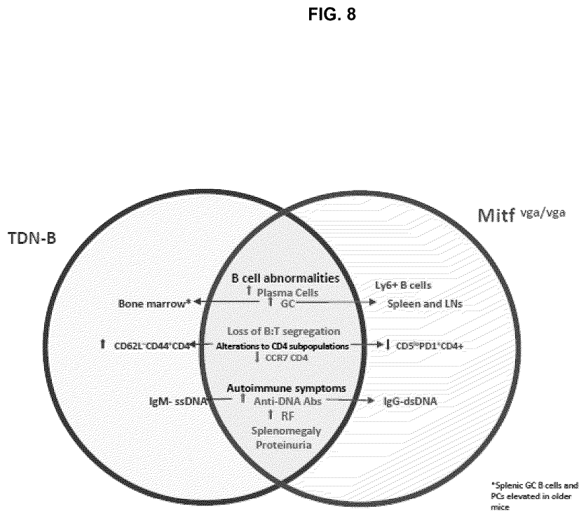

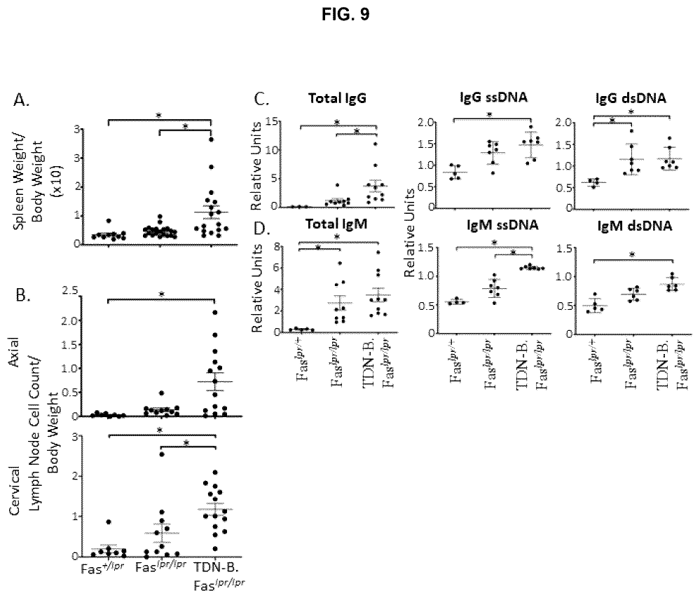

As another way to assess the significance of the autoimmune related phenomena, we asked whether Mitf/MiT family inactivation in B cells could influence the characteristics of autoimmunity and autoimmune disease in the established C57BL/6-Faslpr/lpr model for mild lupus. To accomplish this, the TDN-B transgene was crossed onto the C57BL/6-Fas^{lpr/lpr} background, creating the TDN-B.Fas^{lpr/lpr} mouse line. Indeed, TDN-B.Fas^{lpr/lpr} mice appeared to have more pronounced lymphadenopathy and splenomegaly (FIGS. 9A, 9B, and FIG. 8A), and elevated autoantibody titers to certain autoantigens (FIGS. 9C, and 9D), compared to matched Fas^{lpr/lpr} mice. Enumeration of total cells recovered from the axillary and superficial cervical lymph nodes indicated greater numbers per body mass in the TDN-B.Faslpr/lpr cohort compared to matched Faslpr/lpr mice (FIG. 9B). However, flow cytometry analysis showed that the fraction of lymphocytes and B cells were similar (FIG. 8B), suggesting that MiT inactivation did not further amplify the known effects of the Ipr mutation. Neither splenomegaly nor lymphadenopathy were observed in heterozygote C57BI/6-Fas^{lpr/+} mice (Fas^{lpr/+}) at 6 months of age (FIG. 9A and FIG. 9B).

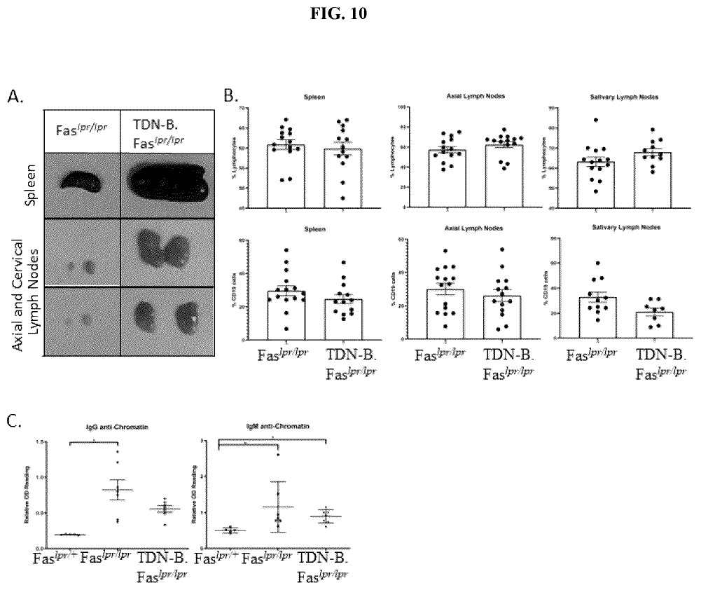

With respect to autoantibodies, total IgG and IgM ssDNA were significantly increased in TDN-B.Fas^{lpr/lpr} mice compared to both wild type and Fas^{lpr/lpr} mice (FIGS. 9C, and 9D), while IgG ssDNA and IgM dsDNA where significantly increased compared to wild type in sera of TDN-B.Fas^{lpr/lpr} mice but not Fas^{lpr/lpr}. In contrast, no significant difference in anti-chromatin antibody titers was observed (FIG. 10C), indicating some selectively in the range of auto-specificities affected by loss of Mitf/MiT function.

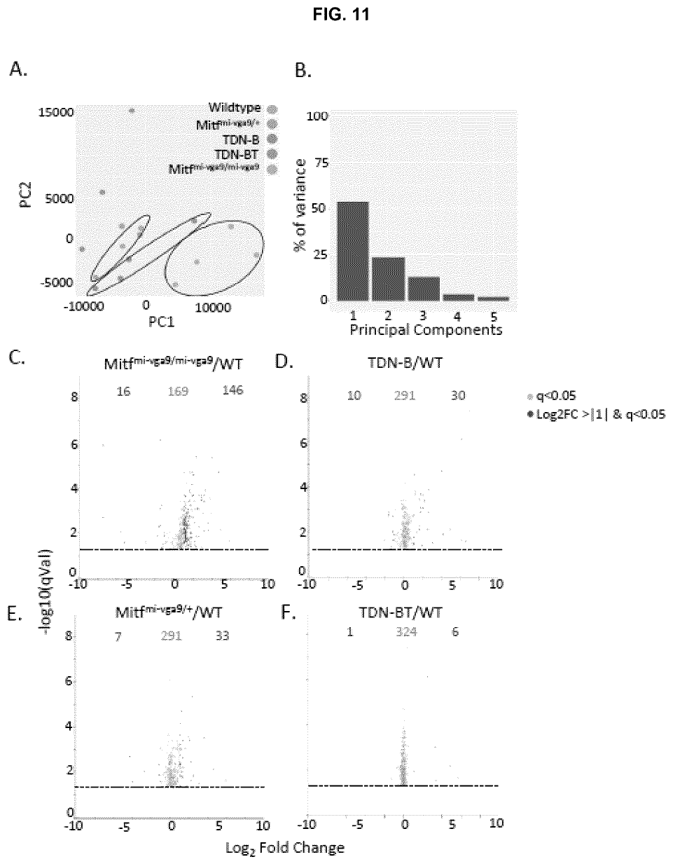

Quantitative transcriptomics of B cells with functional impairments of Mitf and the MiT family transcription factor family: To get a better understanding of how the absence of Mitf and MiT family function impacted the transcriptomic landscape in a way that could explain these abnormalities, we performed RNA-sequencing of ostensibly resting mature B cells isolated through negative selection from wild-type control, Mitf^{mi-vga9/mi-vga9} and TDN-B mice. We also included an analysis of B cells from mice heterozygous for the Mitf null mutation (Mitf^{mi-vga9/+}) as a way to explore Mitf gene dosage effects on B cell target genes, given the impact of Mitf heterozygosity on premelanocytes. Finally, we included B cells from TDN-B/T mice in the analysis to explore the involvement of T cell help in shaping the MiT-deficient transcriptomic landscape. A principal component analysis (PCA) of gene expression was used to summarize the variance of data among the models and plot how each transcript covaries across the samples (FIGS. 11A, and 11B). This PCA was completed based on purified B cell samples from the five genotypes studied and shows segregation of wild type, TDN-B and Mitfmi-vga9/mi-vga9 samples. When comparing MiT functional impairment and wild type B cells, a subset of these transcripts was differentially expressed with a log 2 fold change of <−1 or >+1. The significant cut off was defined by the likelihood ratio q<0.05 among all models, and the fold change is determined by the Wald test between each pairwise comparison (FIGS. 11C-11F, indicated in red).

Summary of differentially expressed transcripts. Upregulated; Table 1A and Downregulated Table 1B genes based on likelihood ratio q<0.05 and Log2 fold change >|1| in each Mitf/MiT family impairment mouse model (TDN-B, TDN-BT, Mitf^{mi-vga9/mi-vga9}, and Mitf^{mi-vga9/mi-vga9}) B cells relative to wildtype B cells.

TABLE 1A

| Upregulated | | | | |
|---|---|---|---|---|
| Genes | Mitfmi- | | | |
| (q < 0.05 & | vga9/mi- | | Mitfmi- | TDN- |
| Log2 > 1) | vga9 | TDN-B | vga9/+ | BT |
| Baiap2 | x | x | x | x |
| Socs6 | x | x | x | x |
| Ighg2c | x | x | | |
| Gcsam | x | x | | |
| Tox2 | x | x | | |
| Nuggc | x | x | | |
| Fam101a | x | x | | |
| Aicda | x | x | | |
| Ighg2b | x | x | | |
| Ackr4 | x | x | | |
| Mef2b | x | x | | |
| S1pr2 | x | x | | |
| H1fx | x | x | | |
| Bmpr1a | x | x | | |
| Mybl1 | x | x | | |
| Rgs13 | x | x | | |
| Chst1 | x | x | | |
| Parm1 | x | x | | |
| Bcat1 | x | x | | |
| Dnm3 | x | x | | |
| Nt5e | x | x | | |
| Rassf6 | x | x | | |
| Tox | x | x | | |
| Ifit8 | x | | x | |
| Ifit2 | x | | x | |
| Slfn5 | x | | x | |
| Rsad2 | x | | x | |
| Lyg6c2 | x | | x | |
| Pydc3 | x | | x | |
| Ighv1-69 | x | | x | |
| Ifi44 | x | | x | |
| Oas1g | x | | x | |
| Slfn1 | x | | x | |
| Gm14446 | x | | x | |
| Ifit1 | x | | x | |
| Mx1 | x | | x | |
| Irf7 | x | | x | |
| Oas3 | x | | x | |
| Usp18 | x | | x | |
| Oas1a | x | | x | |
| Ly6a | x | | x | |
| Rtp4 | x | | x | |
| Ifi2712a | x | | x | |
| Usp40 | | x | x | x |
| Tfe3 | | x | | x |
| Pdgfc | | x | | |

TABLE 1B

| Downregulated | | | | |
|---|---|---|---|---|
| Genes | Mitfmi- | | | |
| (q < 0.05 & | vga9/mi- | | Mitfmi- | TDN- |
| Log2 < 1) | vga9 | TDN-B | vga9/+ | BT |
| Gm18752 | | x | | |
| Oas1g | | x | | |
| Pdgfc | x | | | X |
| Usp40 | x | | | |
| Pkd1I1 | x | x | x | |
| Usp40 | x | | | |
| Nnt | x | | x | |
| Camk2b | x | | x | |
| 9030025P20Rik | x | | x | |
| Cpne4 | x | | x | |

TABLE 2

| Mouse | MiT | Cell Type | Previously Published |
|---|---|---|---|
| Models | Inhibition | Affected | features |
| mi/mi | MiT family | Hematopoietic | Increased plasma |
| chimeras | | cells | cells, anti-DNA |
| Lin et al., | | | autoantibodies, and |
| 2004 | | | rheumatoid factor |
| | | | Increased Irf4 |
| | | | mRNA expression in- |
| | | | vitro |
| | | | No direct Mitf |
| | | | binding site identified |
| | | | on Irf4 promoter |
| | | | through EMSA |
| TDN-B | MiT family | B cells | |
| TDN-BT | MiT family | B and T cells | CD40L impairment |
| | | | with MiT inhibition in |
| | | | T cells |
| | | | Huan et al., 2006 |
| Mitf^{mi-vga9/mi-vga9} | Recessive | All cells | Albino mice |
| | Mitf-null | | No previously |
| | mutation, | | studied or described |
| | with no | | lymphocyte |
| | effect on | | abnormalities |
| | other MiT | | (citation) |
| | family | | |
| | members | | |
| Mitf^{mi-vga9/+} | Heterozygate | All cells | Increased type I |
| | Recessive | | interferon signature |
| | Mitf-null | | in melanoblasts |
| | mutation | | (citation) |

Table 2 above provides a summary of mouse models in RNA-Seq experiments. Summarizes MiT family inhibition, cell type affected, and previously published features of each model, including TDN-B, TDN-BT, Mitfmi-vga9/mi-vga9, Mitfmi-vga9/+. Summary of the mi/mi chimera from Lin et. al. 2004 is also included.

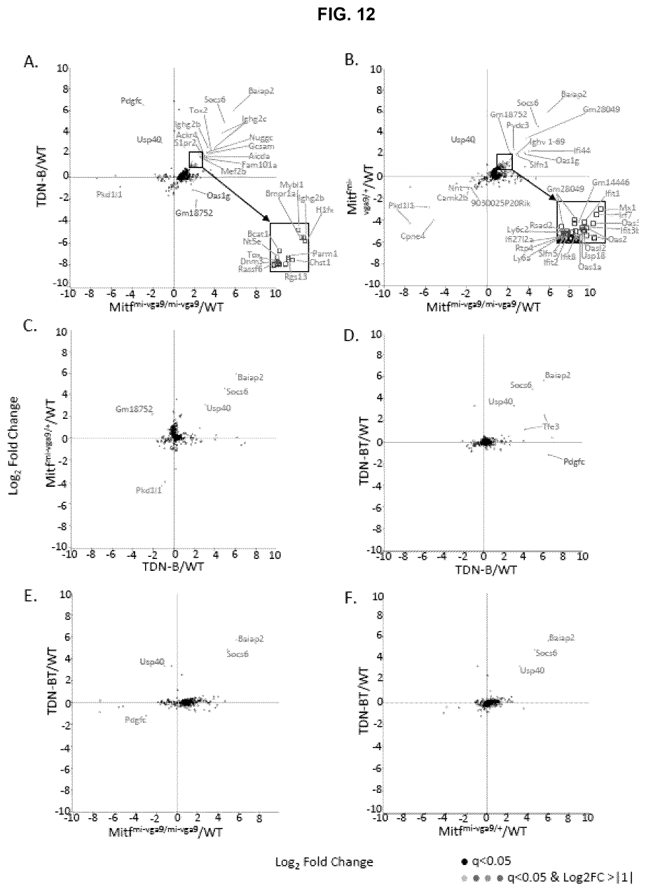

The 331 transcripts that meet the likelihood ratio significance cut off (q<0.05) were plotted to compare relative expression of transcripts across TDN-B, TDN-B/T, Mitf^{mi-vga9/mi-vga9}, and Mitf^{mi-vga9/+} models against wild type expression (FIGS. 12A-F). FIG. 12A provides a scatterplot of significantly differentially expressed transcripts, plotting relative expression of TDN-B against Mitf^{mi-vga9/mi-vga9} B cells (FIG. 12A). Colored and labeled transcripts are those that also have a log 2 fold change greater than one. Relative expression in Mitf^{mi-vga9/+} against Mitf^{mi-vga9/mi-vga9} (FIG. 12B), Mitf^{mi-vga9/+} against TDN-B (FIG. 12C), TDN-B/T against TDN-B (FIG. 12D), TDN-B/T against Mitf^{mi-vga9/mi-vga9} (FIG. 12E), and TDN-B/T against Mitf^{mi-vga9/mi-vga9} (FIG. 12F) are also plotted. The expression of genes known to be involved with germinal center organization and development were shown to be significantly increased in both TDN-B and Mitf^{mi-vga9/mi-vga9} B cells and include Aicda, Rgs13(25), S1pr2(26), Mef2b(27), and Nuggc(28) (FIG. 12A). In contrast, genes shown to be induced in early plasma cells (Oas3 and Oasl2) and plasmablasts (Irf7, Ifit1, ifi44), as well as those related to type I interferon responses, were shown to be significantly increased in both Mitf^{mi-vga9/+} and Mitf^{mi-vga9/mi-vga9} B cells (FIG. 12B), but not in TDN-B B cells. Table 1 highlights 25 transcripts differentially expressed in both TDN-B and Mitf^{mi-vga9/mi-vga9} B cells, including many previously identified as important for germinal center function. Few transcripts were differentially expressed in B cells of the other MiT functional deficiency models when compared to wild type. Only significantly differentially expressed transcripts, defined by their likelihood ratio q<0.05, are indicated in FIG. 12.

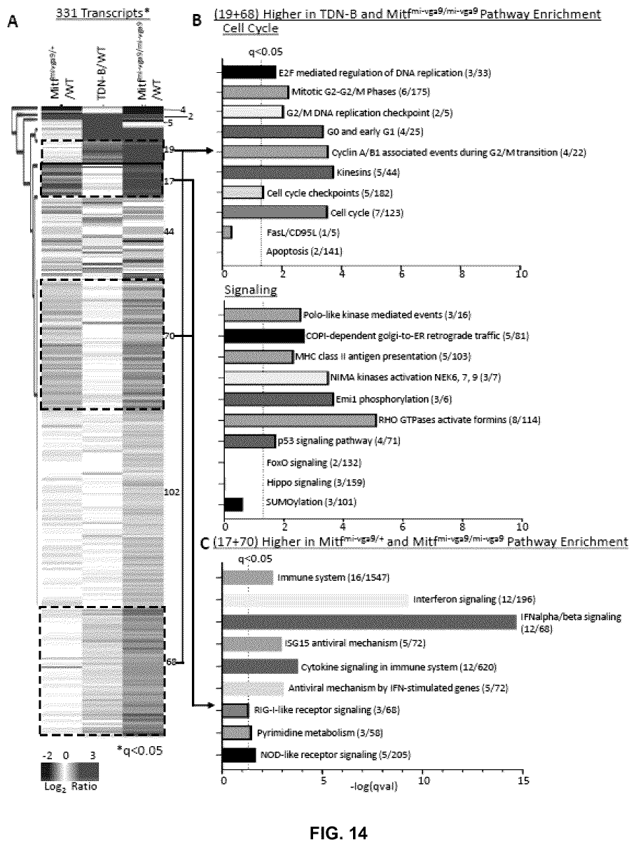

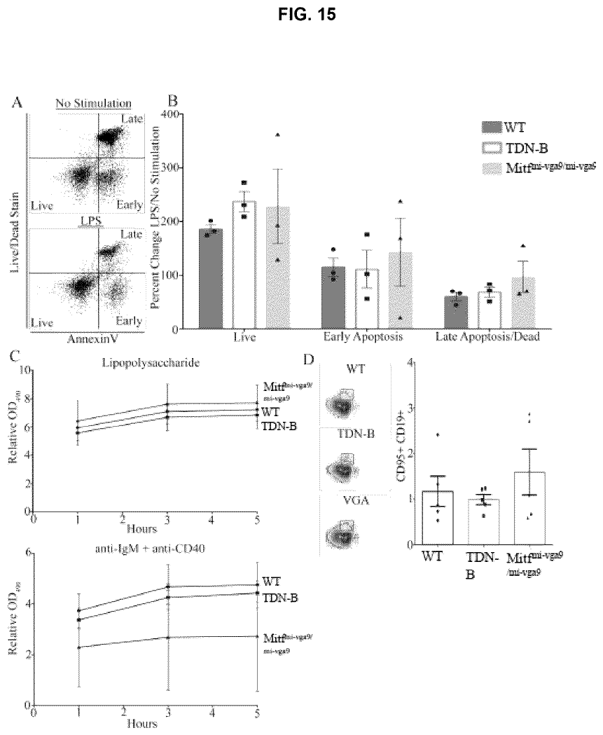

To systematically relate biological processes to corresponding representative transcripts that might be enriched among those differentially expressed, we applied hierarchical clustering analysis across all models (WT, TDN-B, TDN-B/T, Mitf^{mi-vga9/+}, and Mitf^{mi-vga9/mi-vga9}) with the 331 transcripts significantly differentially expressed (likelihood q<0.05) (FIG. 14A). Subsequent pathway enrichment analysis of clusters was completed using Enrichr of clusters with increased expression (FIGS. 14A-14C). Of the cluster with 44 transcripts with decreased expression in TDN-B, TDN-B/T, and Mitf^{mi-vga9/mi-vga9} models compared to wild type, there were no significant pathway enrichments by Fisher's exact test (q<0.05) based on Reactome pathway analysis. In concordance with evidence for increased B cell activation and GCs in Mitf^{mi-vga9/mi-vga9} and TDN-B mice, pathways relevant to activated B cell proliferation, including cell cycle, DNA replication, phosphorylation, kinase activation, and antigen presentation, were enriched in two clusters with 19 and 68 genes in each, with higher expression in TDN-B and Mitf^{mi-vga9/mi-vga9} B cells compared to controls (FIG. 14B). Consistent with elevated Ly6A/E expression on Mitfmi-vga9/mi-vga9 B cells detected by flow cytometry, Ly6a transcript expression was also significantly increased by RNAseq in Mitfmi-vga9/mi-vga9 B cells. Further, pathways relevant to interferon signaling, cytokine signaling, and metabolism were enriched in two clusters of 17 and 70 genes each, with higher expression in Mitf^{mi-vga9/+} and Mitf^{mi-vga9/mi-vga9} B cells compared to TDN-B and wild-type controls (FIG. 14C). In contrast, pathways including apoptosis known to be regulated by Mitf and MiT family members in other cell types were not significantly enriched in these Mitf^{mi-vga9/+} and Mitf^{mi-vga9/mi-vga9} B cell RNAseq clusters. Consistent with these findings, significant differences in apoptosis of naïve B cells in culture were not observed through flow cytometric and LPS or IgM+CD40 stimulation functional assays (FIG. 15).

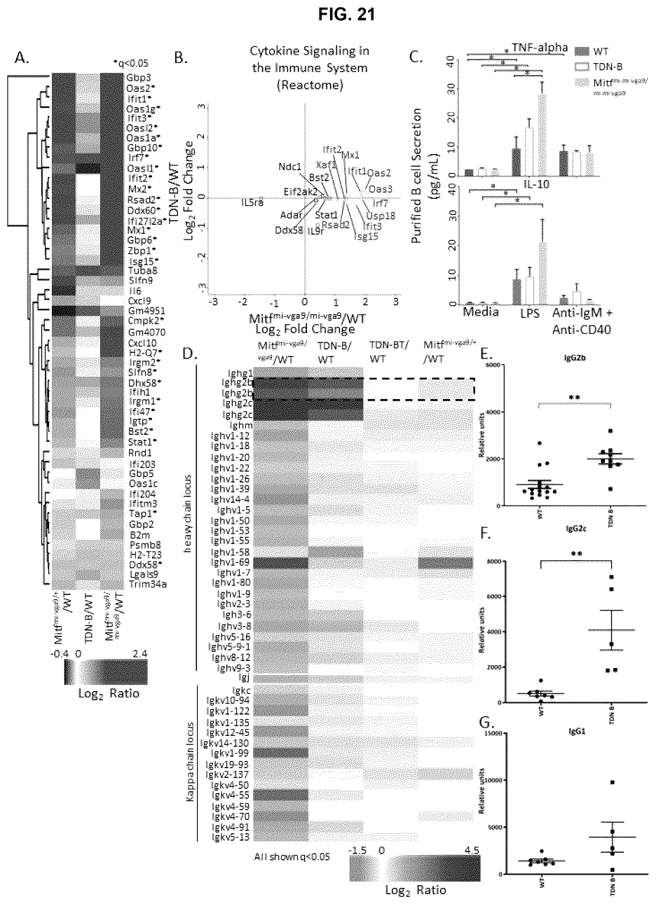

Using RNAseq, Harris et al. (2018) reported the upregulation of 55 genes of innate immune signaling in Mitf^{mi-vga9/+} melanocyte stem cells, supporting a role of Mitf in innate immune response. Among those 55 genes, we found that 30 genes were similarly increased in Mitf^{mi-vga9/+} and Mitf^{mi-vga9/mi-vga9} B cells (FIG. 21A, marked by asterisk*). However, these changes were not observed in TDN-B B cells (FIG. 21B), a finding consistent with the flow cytometry results for Ly6 expression (FIG. 3F). Similarly, multiplex immunoassays showed elevated expression of TNF-alpha in Mitfmi-vga9/mi-vga9 B cell culture supernatants after LPS stimulation, but not in TDN-B B cell supernatants (FIG. 21C). No differences were seen in the production of IL-10 between groups with either LPS or anti-IgM+ anti-CD40 stimulation. The RNAseq data indicated that in both TDN-B and Mitfmi-vga9/mi-vga9 B cells, there was increased expression of immunoglobulin genes from the heavy chain and kappa chain locus, especially IgG2b and IgG2c (FIG. 21D). Consistent with this finding, serum titers of TNP-specific IgG2b and IgG2c, but not IgG1, were significantly increased in the sera from TDN-B mice seven days after immunization with TNP-ficoll when compared to sera from wild type mice (FIGS. 21E, 21F, and 21G).

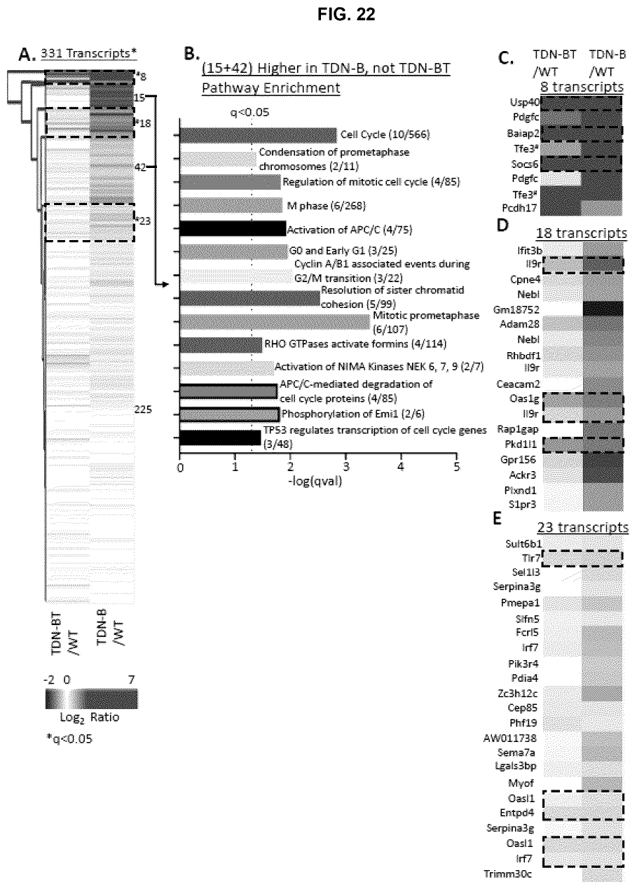

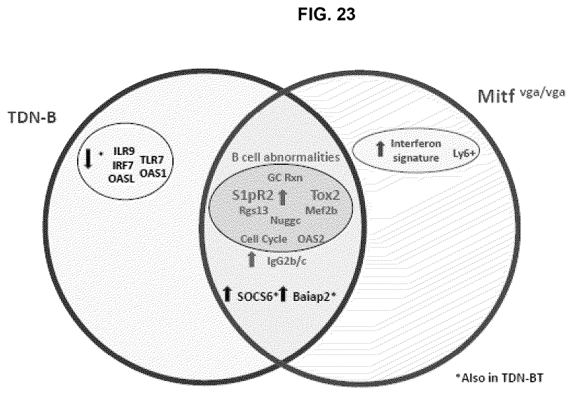

Comparison of TDN-B and TDN-B/T B cell transcripts helps delineate T helper cell dependent pathways. We next sought to compare gene expression patterns in TDN-B and TDN-B/T B cell through heat map visualization to assess the B cell transcripts influenced by T cell help, which is impaired due to TDN expression in T cells in the TDN-B/T line. While most transcripts showed minimal differential expression compared to wild type (FIG. 22A, Cluster 225), two clusters enriched with cell cycle, cell division, and kinase activation signaling pathways showed increased transcripts in TDN-B, but not TDN-B/T B cells (FIG. 22A, Clusters 15 and 42), suggesting an effect of T cell help on MiT function in B cells(FIG. 22B). The T cell independent MiT activities were revealed by the finding of commonly changed transcripts in TDN-B and TDN-B/T B cells as compared to wild type (Clusters 8,18,23, FIG. 22A and FIGS. 22C-22E). These transcripts include highly upregulated Usp40, Baiap2, and Socs6 (Cluster 8) and downregulated II9r, Pkd111, TIr7, Oasl1, Entpd4, and Irf7 (Cluster 18 and 23). Notably, Tfe3 was expected to be higher in both models than in WT because the TDN gene was constructed from Tfe3 cDNA sequences, and which served to independently validate the RNAseq. FIG. 23 summarizes gene expression changes observed in TDN-B and Mitfmi-vga9/mi-vga9 B cells.

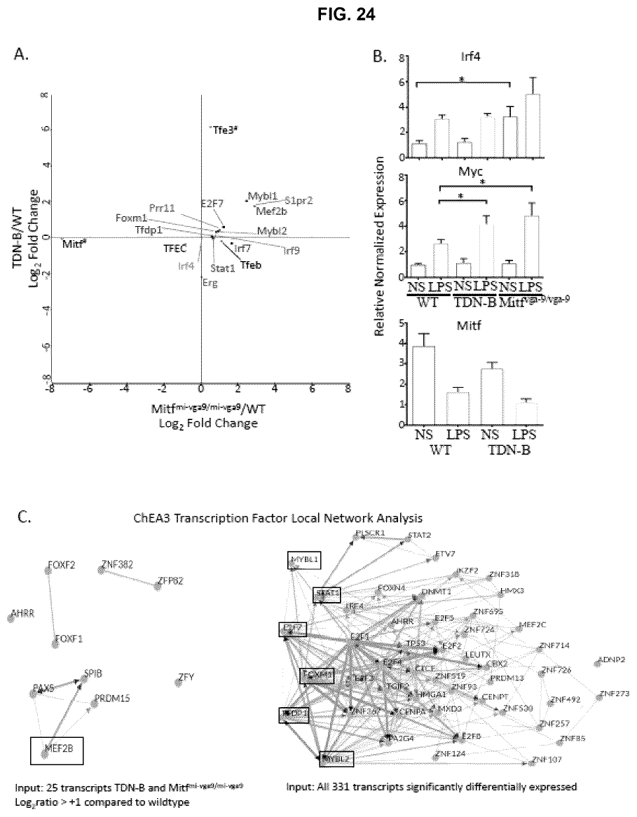

Defining differentially expressed transcripts in TDN-B and Mitfmi-vga9/mi-vga9 B cells. As shown in FIG. 24A, an unexpected finding was that Irf4 transcript abundance was no different than wild type in Mitf^{mi-vga9/mi-vga9} or TDN-B B cells, residing at almost 0,0 in each comparison (FIG. 24A, orange). Consistent with that finding, RT-qPCR results showed no statistically significant difference in Irf4 mRNA between control and TDN-B cells, whereas there was only a 2-3-fold increase of Irf4 mRNA in unstimulated Mitfmi-vga9/mi-vga9 B cells compared to WT or TDN-B B cells (FIG. 24B). In contrast, LPS stimulation increased Irf4 expression about 2-fold in WT and TDN-B B cells, but not in Mitfmi-vga9/mi-vga9 B cells. This is in contrast to the dramatic increase of basal (>4 fold) and inducible (>5 fold) Irf4 expression in Mitfmi/mi B cells reported by Lin et al. (1). We believe that this difference possibly reflects the effects of MiT inactivation in white blood cells other than B cells in the Mitfmi/mi HSCT model, which is supported by Lin et al.'s conclusion that the binding site for Mitf on Irf4 promoters could not be found (1). Interestingly, genes that have been found in prior studies to be directly bound by Mitf, such as Prr11, S1pr2, Erg, and Irf9, which were identified by a ChIP-Seq analysis of mouse mast cells, were differentially expressed in TDN-B and Mitfmi-vga9/mi-vga9 B cells (FIG. 24A, green). In contrast to Irf4, the abundance of Myc transcripts was greater in TDN-B and Mitfmi-vga9/mi-vga9 B cells compared to wild type after 24 hours in culture with LPS stimulation (FIG. 24B), consistent with the hypothesis that mitogenic stimulation may drive increased proliferation in B cells that have impaired Mitf function, as compared to wild type B cells.

Given that transcription factors were among those differentially expressed in Mitf/MiT-deficient B cells, we sought to predict what transcription factors other than Mitf/MiT might participate in regulation of the differentially expressed transcripts. We performed a transcription factor enrichment analysis using ChIP-x enrichment analysis 3 (ChEA3), a gene-set enrichment analysis tool tailored to test if query gene-sets are enriched with genes that are known putative targets of transcription factors. As shown in FIG. 24C, one of the 10 Top-ranked transcription factors predicted by ChEA3 analysis was Mef2b, which was found significantly increased in TDN-B and Mitfmi-vga9/mi-vga9 B cells compared to wild type. Therefore, Mef2b is a possible downstream target that is normally repressed by Mitf in B cells (FIG. 24C). In addition to Mef2b, Mybl1, Mybl2, Foxm1, E2f7, Tfdp1, and Stat1 were also predicated by ChEA3 within the 50 Top-ranked transcription factors associated with the 331 transcripts significantly differentially expressed, all of which were also significantly differentially expressed in TDN-B and Mitfmi-vga9/mi-vga9 B cells compared to wild type (FIG. 24A, Blue). FIG. 23 provides a summary Venn diagram of phenotypic and select transcription abnormalities observed in TDN-B vs. Mitfmi-vga9/mi-vga9 mice.

Genes differentially expressed with Mitf and MiT family impairment are also differentially expressed in normal human B cell subsets. We next sought to consider how B cell gene expression patterns observed in our Mitf and MiT family deficient mouse models relate to human B cell biology. Using SAM (Significance Analysis of Microarray) multiclass analysis, Kassambara et al. (2015) defined 9303 genes significantly differentially expressed in different subsets of human B cells including naïve B cells, memory B cells, centroblasts, centrocytes, preplasmablasts, plasmablasts, early plasma cells, and bone marrow mature plasma cells.

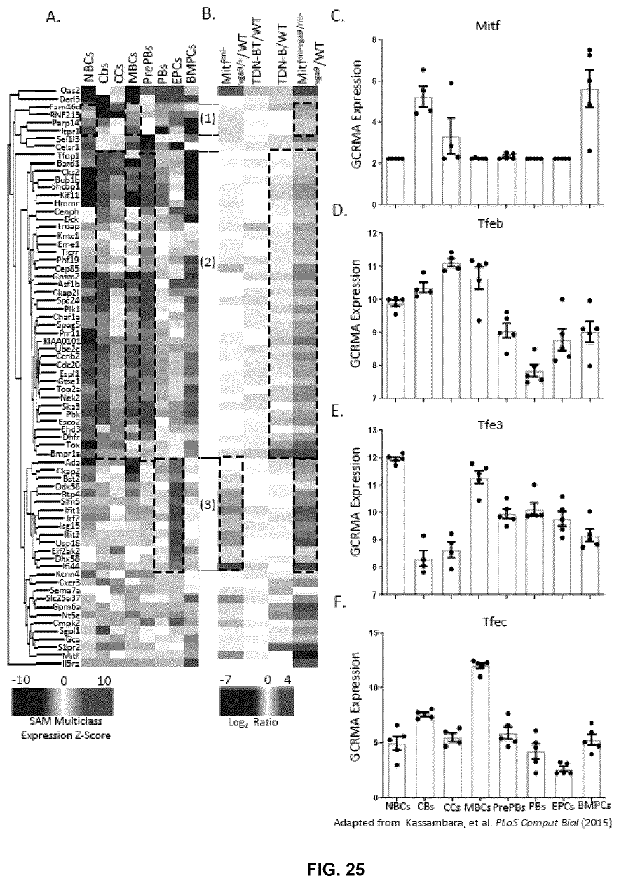

Interestingly, by comparing the expression data from Kassambara et al., 2015(13) to our mouse gene datasets, we observed that 72 genes were significantly differentially expressed in both normal human B cell subsets and mouse B cells with Mitf and MiT family impairment (FIG. 25A and FIG. 25B). Specifically, transcripts increased in human naïve B cells and memory B cells were also increased in Mitf^{mi-vga9/mi-vga9} mouse B cells; transcripts that were increased in human centroblasts, centrocytes, and pre-plasmablasts were also increased in Mitf^{mi-vga9/mi-vga9} and TDN-B mouse B cells, and transcripts that were increased in human plasmablasts and early plasma cells were also increased in Mitf^{mi-vga9/+} and Mitf^{mi-vga9/mi-vga9} mouse B cells (FIG. 25A and FIG. 25B, dotted borders).

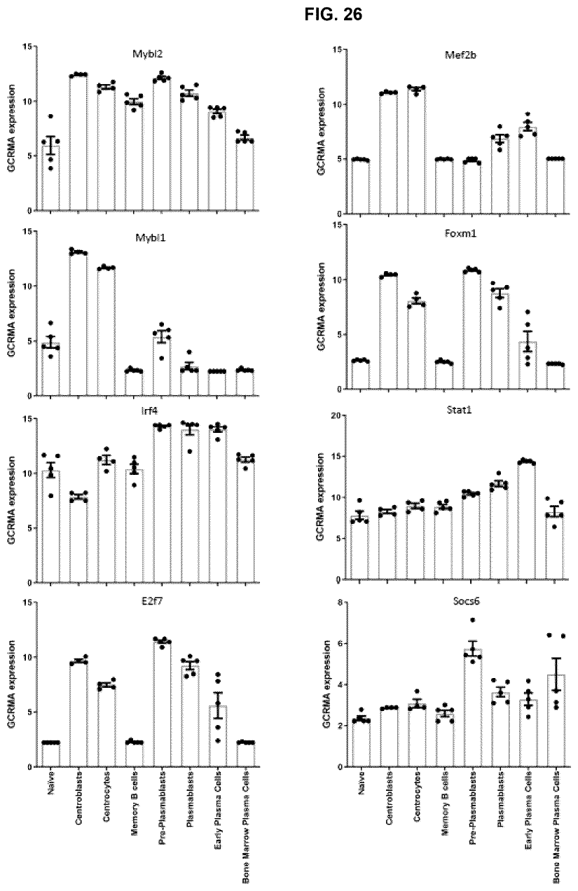

Distribution of expression of MiT family members across B and plasma cell developmental stages are also shown (FIG. 25C-25F). All four MiT family members were significantly differentially expressed among these clusters. For instance, among the MIT family members, TFE3 and TFEB were expressed in resting mature B cells, while Mitf and TFEC were not. In contrast, TFEC expression was highest in memory B cells. TFE3 and TFEB were also present in memory B cells, whereas Mitf was not detected in this subset (FIG. 25). FIG. 26 shares the distribution of expression of other significantly differentially expressed genes in MiT mouse model B cells (MyblI1, Mybl2, Mef2b, Foxm1, Stat1, E2f7, and Socs6), as well as Irf4. Overall, this meta-analysis shows that genes differentially regulated in naïve B cells from our Mitf- and MiT-deficient mouse models have similar expression patterns corresponding to specific developmental stages and mature subsets of human B cells, supporting a regulatory role of Mitf and MiT in B cells and the importance of Mitf target genes in human B cell development.

Although the study of Mitf in B cells started over three decades ago, how Mitf exerts its function through downstream target genes and pathway modulation in B cells has remained obscure. Our study emphasizes and clarifies the importance of Mitf to B cell homeostasis, activation, and tolerance, using distinct, but related Mitf-deficient mouse models, which complement the original HSCT model that first identified this key relationship. These models also provide more detailed evidence of the range of abnormalities in lymphocyte compartments caused by lack of Mitf function, either in B cells or systemically, and provide new data about the gene expression program Mitf influences to restrain B cell activation and entry into the germinal cell/plasma cell (GC/PC) developmental program. Unexpectedly, these findings also challenge a long-standing model that proposed that Mitf restrains GC and PC development through repressing Irf4 expression. In sum, the data presented here, through complementary models of Mitf functional impairment, are consistent with a model in which Mitf regulates genes in a B cell intrinsic manner that is further influenced through T helper cell dependent process, in which dysregulation caused by Mitf inactivation drives B cell subset abnormalities and causes splenomegaly, elevated auto-antibodies, and proteinuria.

Immune abnormalities in different models for Mitf/MiT inactivation point to a pivotal role of Mitf. Comparing findings among the different models for Mitf/MiT inactivation provided a systematic approach to resolve differences between the effects of systemic versus B cell intrinsic functions. Also, these models begin to differentiate null versus trans-dominant effects, the latter of which may influence activities of other MiT family members and aspects of cell metabolism they might uniquely control. From this vantage point, it is noteworthy that all three models—TDN-B, Mitf^{mi-vga9/mi-vga9}, and the Mitf^{mi/mi} HSCT chimera—have an increased spontaneous frequency of plasma cells, produce elevated serum immunoglobulins, and elevated autoantibodies, including rheumatoid factor and anti-DNA. This supports the validity of using all three models to explore B cell effects of Mitf and MiT family functional inactivation and dissect which genes are influenced under which set of circumstances.

Other related, but previously undocumented spontaneous abnormalities in TDN-B and Mitf^{mi-vga9/mi-vga9} mice not described in the HSCT model, include splenomegaly, mild proteinuria, and changes to splenic lymphoid architecture. Changes to lymphocyte populations included elevated PCs/GCs in bone marrow and lymph nodes, increased frequency of Ly6A/E+Ly6C+ B cells, and changes in the frequencies of CCR7+ and CD62LloCD44hi CD4 T-cell subsets. Additionally, transcription upregulation of genes linked to germinal center regulation, plasma cell differentiation, and B cell activation were upregulated in TDN-B and Mitf^{mi-vga9/mi-vga9} mouse B cells, including Mef2b(32), Aicda(24), Rgs13(25), S1 pr2(26), Mef2b(27), Nuggc(28), IgG2b, and IgG2c, among others (Table 1). The upregulation of these genes and the phenotypic changes observed suggest there is a breach in B cell tolerance in TDN-B and Mitf^{mi-vga9/mi-vga9} mice whereby Mitf and MiT family members' loss-of-function permits increased transcriptional expression of genes that promote increased B cell activation and dysregulation of the germinal center/plasma cell developmental program. Although many of these phenotypes were not described in the adoptive transfer HSCT system, the present study evaluated mouse B cells at 3-8 months of age, a time period which may have allowed the emergence of phenotypes that may require particular developmental contexts and/or environmental exposures to evolve.

Comparisons of the TDN-B and Mitf^{mi-vga9/mi-vga9} models to TDN-B/T mice suggested that many, but not all, B cell abnormalities were T-helper cell dependent. The discrete B cell—T cell partition observed in splenic follicles of un-immunized wild type mice was disrupted in TDN-B and Mitfmi-vga9/mi-vga9 mice, but remained intact in TDN-B/T mice, suggesting that B cell intrinsic processes that control partitioning are regulated by Mitf. Although disorganization of follicles correlated with the elevated frequency of GC and plasma cells, further studies are required to define the relationship between these phenomena and the underlying mechanisms responsible.

Importantly, these phenomena appear independent of a generalized elevated type I IFN signature caused by systemic Mitf deficiency that was evidenced by expression of Ly6 on B lymphocytes from Mitfmi-vga9/mi-vga9, but not TDN-B mice. Previously, an altered microenvironment, proposed to be due to increased production of type 1 interferons, was suggested by the effects observed on melanocyte progenitor stem cells in Mitfmi-vga9/+ mice. Although both Mitf^{mi-vga9/mi-vga9} mice and TDN-B mice produced anti-DNA antibodies, this elevated type 1 interferon gene signature might help explain why anti-dsDNA antibody was produced in Mitfmi-vga9/mi-vga9 mice, whereas anti-ssDNA was made in TDN-B mice. Greater evidence of dysregulated B cell activation in vitro was also observed in Mitfmi-vga9/mi-vga9 mice, specifically, enhanced secretion of TNF-alpha in vitro by Mitf^{mi-vga9/mi-vga9} B cells and increased c-myc expression in TDN-B B cells after LPS stimulation. However, further work is needed to determine whether the enhanced responsiveness of B cells from Mitf^{mi-vga9/mi-vga9} mice to mitogenic stimuli was intrinsic to Mitf deficiency itself, or due to cell interactions such as T-cell dependent signaling or development in an Mitf-deficient cytokine milieu that persisted in culture. Further work is also required to determine if similar increased proliferative signatures are present in Mitf-inactivated B cells obtained from microenvironments that lack T cell help.

MITF-mediated regulation of B cell responses: Our findings further establish Mitf as a key player in regulating B cell responses through restraining B cell activation and PC development. Interestingly, Mitf seems to act as a major repressor of gene expression in B-cells. Unexpectedly, our RNAseq analysis showed that Irf4 expression was unchanged in ex-vivo B cells from all four models of Mitf inactivation that we used (Mitf^{mi-vga9/mi-vga9} TDN-B, TDN-B/T, Mitf^{mi-vga9/+}), a finding largely confirmed in cultured TDN-B and Mitf^{mi-vga9/mi-vga9} B cells via RT-PCR. After 20-24 hour culture, basal and LPS-induced levels of Irf4 were modestly (-2-fold) higher than wild-type in Mitf^{mi-vga9/mi-vga9} B cells, whereas Irf4 levels in TDN-B B cells were comparable to wild type. This is in contrast to the Mitfmi HSCT model in which orders of magnitude increases in Irf4 expression were observed in Mitf deficient B cells. Our findings do not provide evidence to support a role for Mitf as a repressor of Irf4 in B cells. Although it is not clear yet what the underlying reasons are for these differences in Irf4 expression in different model systems, given that in the Mitfmi HSCT model the entire MiT family is also potentially inactivated in hematopoietic cells other than B cells, upregulation of Irf4 transcription in the Mitfmi HSCT model by non-B cell factors cannot be excluded. Interestingly, in melanocytes Mitf is a direct activator of Irf4, indicating cell type specificity in how this transcription factor network is regulated.

Transcription factor enrichment analysis demonstrated that Mef2b is a Top-ranked transcription factor that is predicted to be part of the transcription factor network responsible for regulating gene expression in both TDN-B and Mitfmi-vga9/mi-vga9 naïve B cells. Since Mef2b is a known transcription factor that regulates B cell differentiation to germinal center B cells and is included in a network with other transcription factors known to regulate B cell to plasma cell differentiation (FIG. 24C), the observed transcriptional regulation of Mef2b by Mitf in B cells could define an alternate pathway to that proposed by Lin et al. 2004 linking Mitf from B cell to plasma cell development. Additional alternate pathways are supported by Top-ranked transcription factor network analysis of the 311 genes with significant differential expression in B cells across Mitf and MiT family impairment models. Overall, differential transcription factor expression in TDN-B and Mitfmi-vga9/mi-vga9 B cells supports the existence of a network of gene transcription that is B cell autonomous, correlating with B cell activation and regulation. Select transcription factors known to directly regulate B cell to plasma cell differentiation, including Irf4, E2f1, and Mef2c, were not significantly differentially expressed in TDN-B and Mitfmi-vga9/mi-vga9 B cells, but were included in transcription factor network mapping, supporting indirect, perhaps non-autonomous B cell roles affected by Mitf and MiT family members.

Changes in the Mef2b network are consistent with evidence of germinal center program dysregulation given that the molecules in this network have known roles in B cell differentiation and germinal center processes. Particularly, Aicda (AID) and Nuggc (also known as SLIP-GC) have been shown to have similar expression patterns in germinal center B cells: Aicda is necessary for somatic hypermutation (SHM) and class switch recombination; and increased Aicda expression in TDN-B and Mitfmi-vga9/mi-vga9 supports that Mitf is a negative regulator of Aicda. Further studies are required to determine whether Aicda has promoters directly regulated by Mitf.

Another gene repressed by Mitf, Rgs13, is known to regulate cell cycle and germinal center specific genes. Rgs13 regulates extra-follicular plasma cell generation, germinal center size, and germinal center B cell numbers. The finding that the expression of Rgs13 is increased upon Mitf loss-of-function indicates perturbations in naïve B cell homeostasis and may help explain the spontaneous disorganization of many follicles observed in TDN-B and Mitf^{mi-vga9/mi-vga9} mice. Interestingly, Rgs13, and another differentially expressed gene, S1pr2, are both involved in germinal center confinement of B cells. S1pr2, through binding with S1P, defines a gradient that restricts B cell movement within the germinal center. Further studies are needed to discern whether and how perturbation of Rgs13 and S1pr2 expression through Mitf inactivation could explain the observed changes in B and T cell localization.

Socs6 and Baiap2 emerge as two new novel targets of Mitf-mediated repression. The expression of both genes is dramatically increased across all four models of Mitf inactivation (Mitf^{mi-vga9/mi-vga9}, TDN-B, TDN-B/T, and Mitf^{mi-vga9/+}), but their specific mechanisms of involvement in B cell regulation requires further investigation. Socs6 regulates cytokine signaling, including Kit receptor functions, and given that Kit regulates lymphocyte development, it may contribute to the lymphocyte disorganization and/or disrupted B cell homeostasis observed in this study. Baiap2, also known as IRSp53, is a scaffold/adaptor protein that regulates membrane dynamics with Cdc42(39), which may affect B cell activation and differentiation. However, given that splenomegaly, RF, and elevated urine protein were not observed in all lines, the increased expression of these genes is either not relevant or not sufficient to cause these B cell abnormalities. Regardless, the changes driven by Socs6 and Baiap2 warrant further study.

Interestingly, a small number of transcripts, including Usp40, Pkd111, Pdgfc, and Cpne4, have significantly decreased relative expression (Irt q<0.05, FC<−1) in Mitf^{mi-vga9/mi-vga9} B cells compared to wild type. Whether the decreased expression of these transcripts, particularly Pdgfc and Usp40, contributes to primary follicle disorganization in Mitfmi-vga9/mi-vga9 mice remains to be determined. Given that these changes are unique to the Mitf-null model, the relationship of these genes to Mitf function in B cells and the influence of their interactions with other MiT members and other cell types remain unclear. One consideration includes further study into the downstream effects on apoptosis that may result from decreased expression of Usp40 and Pdgfc in Mitf-null model B cells. Further study into Usp40 and Pdgfc is especially intriguing to consider because it is also unclear why Pdgfc is increased in TDN-B only, but low in all other models. Pdgfc has been reported to enhance Erk activation and adhesion-independent growth of pre-B cell lines, but its role in naïve and activated B cells is still unknown. Whether or not the increased Pdgfc in TDN-B B cells, but not Mitf^{mi-vga9/mi-vga9} B cells, is linked to increased pre-B cell proportions in TDN-B mice only (FIG. 5) requires further investigation. Usp40, on the other hand, was increased in TDN-B, TDN-B/T, and Mitf^{mi-vga9/+}, but was absent from Mitfmi-vga9/mi-vga9 B cells. We theorize that Usp40 may be influenced by Mitf gene dosage and may also be impacted by the complete loss-of-function of Mitf in non-B cells. Further work is needed to determine the importance of these findings to B cell biology and how they are regulated by the members of the MiT family.

In other cell types, Tfeb and Mitf have been defined as master regulators of autophagy and Mitf has been shown to drive endolysosomal biogenesis. However, our study did not discover any changes in the expression patterns of genes related to autophagy, mitophagy, and lysosomal biogenesis. These include mTOR, Atg5, Atg7, all V-type proton ATPase subunits, RagA,B,C, and D, and LAMP1 and 3. Mitf has also been shown to regulate genes involved in reactive oxygen species, lysosome biogenesis/autophagy important for antigen presentation, PC development, and B cell activation/GC responses, as have Tfe3 and Tfeb. An important direction for future studies will be to analyze how Mitf-dependent pathways interact with these processes.

No changes were observed in the expression of CD84 or Ly108 (Slamf6), both of which are implicated in restraining GC development and preventing the production of lupus autoantibodies. On the other hand, T-Bet (Tbx21), which is associated with age-related ABC B cells, was significantly differentially expressed in Mitfmi-vga9/mi-vga9 B cells compared to wild type and may be a downstream regulator of spontaneous germinal centers that are a source of autoantibodies. However, it does not seem to be responsible for the B cell abnormalities shared by TDN-B and Mitfmi-vga9/mi-vga9 mice since it was not upregulated in TDN-B B cells. In addition, our data indicate that pathways related to apoptosis, FoxO signaling(49), and SUMOlyation(50) were not significantly enriched in our pathway analysis of TDN-B and Mitfmi-vga9/mi-vga9 B cells.

Lastly, prior published data in human cells, including naïve B cells, germinal center B cells, memory B cells, and plasma cells, demonstrated significant differential expression of MiT family members (FIG. 25). Further, many genes differentially expressed among normal human B cell subsets corresponded to those found to be regulated by Mitf in mouse B cells in the present study, justifying future work in human B cells to explore how Mitf and MiT family members contribute to human B cell development and B cell-driven disease. It will also be important to further analyze the B-cell receptor repertoire in TDN-B and Mitfmi-vga9/mi-vga9 B cell and plasma cell populations to explore how that may relate to their abnormal expansion and pathological antibody specificities, and compare those to the B-cell repertoires of SLE patient samples. Given that human memory B cells showed differential expression of MiT family member genes (FIG. 25), and since B cell subsets that may differentiate into germinal center-dependent or germinal center-independent memory B cells (CD19+CD38+GL7+) were significantly increased in Mitfmi-vga9/mi-vga9 mice (FIG. 3D), these data provide direction for future functional studies that focus on the role of MiT members in memory B cell differentiation.

Mitf and autoimmunity: Despite the autoimmune features exhibited by TDN-B and Mitf-null mice, the enhancement of certain autoimmune-associated abnormalities in the TDNB.Faslpr/lpr mice, and the “interferon signature” of systemic Mitf insufficiency, how Mitf relates to lupus and whether Mitf has an involvement in human autoimmune disease remain unclear. Lupus-like symptoms have not been reported in individuals that lack Mitf gene function, but it's not clear it's ever been examined. The interaction of MiT inactivation and the Ipr mutation was a starting point to identify in what possible capacities Mitf/MiT might have such a role and through which pathways, which in turn could be influenced by known autoimmune mechanisms and pathways. The anti-chromatin titers were not affected, and that anti-DNA titers were elevated, seem to indicate some selectivity to self-reactivity influenced by Mitf. It should be noted that there were no discernable perturbations in the expression patterns of genes involved in mediating TLR pathways or other genes known to be dysregulated in other mouse lupus models. However, given that autoimmune symptoms develop with B cell specific loss of function of MiT family members, this warrants further exploration of the relationship of Mitf to B cell tolerance.

As such, Mitf and the novel putative downstream regulated genes presented here will provide new avenues to dissect how B cells interact with their environment, perceive, and interpret signals, and in turn program a response that controls activation and differentiation. From this perspective, future work will focus on how Mitf is positioned to regulate gene expression directly, and which signaling pathways activate/inactivate it in the setting of normal homeostasis, inducible protective immunity, and destructive autoimmunity.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation. All patents, patent applications, and other scientific or technical writings referred to anywhere herein are incorporated by reference herein in their entirety.

Persons having ordinary skill in the art will recognize certain modifications, permutations, additions and sub-combinations thereof. It is therefore intended that the following appended claims hereinafter introduced are interpreted to include all such modifications, permutations, additions and sub-combinations within their true spirit and scope. Those in the art will understand that a number of variations may be made in the disclosed embodiments, all without departing from the scope of the invention, which is defined solely by the appended claims.

What we claim is:

1. A method for treating an autoimmune disease characterized by increased Tfec levels in pathogenic B cells in a subject in need thereof, said method comprising:
administering to said subject a Tfec lowering or inhibiting dose of an agent into said B cells.

2. The method of claim 1, wherein the B cell-mediated autoimmune disease is selected from the group consisting of systemic lupus erythematosus, rheumatoid arthritis, Sjogren's syndrome, and multiple sclerosis.

3. The method of claim 1, wherein the autoimmune disease is systemic lupus erythematosus.

4. The method of claim 1, wherein said agent is a composition comprising a nucleic acid molecule that decreases the functionality of the Tfec protein

5. The method of claim 4, wherein a vector is utilized for delivering the nucleic acid molecule to B cells, and optionally, a pharmaceutical carrier.

6. The method of claim 4, wherein said composition comprises an immunosuppressive and/or immunomodulatory agent to enhance the therapeutic effect.

7. The method of claim 5, wherein the nucleic acid molecule is delivered using a lentiviral vector.

8. A method for modulating B cell homeostasis and tolerance, comprising: i) identifying a subject with impaired Mitf function, ii) introducing a nucleic acid molecule encoding a functional Mitf protein into B cells of the subject, and iii) expressing the Mitf protein in the B cells to regulate gene expression networks implicated in B cell homeostasis and tolerance.

9. A composition for treating autoimmune diseases associated with impaired Mitf function of the method of claim 8, said composition comprising a nucleic acid molecule encoding a functional Mitf protein,

10. The composition of claim 9, wherein a vector is utilized for delivering the nucleic acid molecule to B cells, and optionally, a pharmaceutical carrier.

11. The method claim 8, further comprising: i) identifying candidate target genes regulated by Mitf in B cells; ii) generating a mouse model with a systemic loss-of-function mutation of Mitf or B-cell specific inactivation of the MiT family; iii) performing RNAseq analysis on resting B cells from the mouse model to identify differentially regulated genes; and iv) validating the identified genes as targets for therapeutic intervention in B cell-related autoimmune diseases.

12. The method of claim 8, wherein the nucleic acid molecule is delivered using a lentiviral vector.

13. The composition of claim 9, further comprising an immunosuppressive and/or immunomodulatory agent to enhance the therapeutic effect.

14. The method of claim 8, wherein the identified target genes are used to develop small molecule inhibitors or activators to modulate B cell activation and tolerance.

15. A method for regulating the activity of Tfec transcription factor in B lymphocytes, said method comprises, enhancing the activity of one or more MiT family transcription factors selected from the group consisting of Mitf, Tfeb, and Tfe3.

16. The method of claim 15, wherein the method is for treating a B cell-mediated autoimmune disorder.

17. A method of regulating Mitf and Tfec transcription factors for restoring immune tolerance in autoreactive B cells, comprising: (a) blocking Tfec DNA or RNA gene transcription or protein biosynthesis, respectively, utilizing at least one technique selected from the group consisting of engineered zinc finger proteins, CRISPR-guided base editing, transcription activator-like effector nucleases (TALENs), and RNA-targeted therapeutics using ADARs; or (b) indirectly targeting Tfec protein using small molecules or other scaffolds that inhibit its function or promote its degradation; or (c) increasing Mitf expression and translation by: (i) utilizing engineered zinc finger proteins to activate the Mitf gene promoter, (ii) utilizing CRISPR-guided base editing to introduce beneficial mutations in Mitf gene regulatory regions, or (iii) decreasing proteosomal degradation of Mitf, thereby restoring the ability of Mitf, in conjunction with Tfe3 and/or Tfeb, to maintain immune tolerance in autoreactive B cells.

18. A method of diagnosing a patient exhibiting altered B lymphocyte functionality, comprising: (a) obtaining a biological sample from the patient; (b) isolating B lymphocytes from the sample; (c) measuring expression levels of Tfec, Mitf, Tfeb, and Tfe3 in the isolated B lymphocytes or B cell subsets; and (d) comparing the measured expression levels to established normal ranges for healthy controls and patients with B cell-mediated autoimmune disorders.

19. The method of claim 18, wherein the B cell subsets are selected from the group consisting of double negative 2 B cells, memory B cells, regulatory B cells, aging-associated B cells, and plasmablast B cells.

20. The method of claim 18, wherein measuring expression levels is performed using a technique selected from the group consisting of quantitative PCR, Western blotting, and immunofluorescence.
